(19) United States
(12) Patent Application Publication
(10) Pub. No.: US 2025/0345607 A1
(43) Pub. Date: Nov. 13, 2025

(54) SYSTEMS, DEVICES, AND METHODS FOR ALTERING MIDBRAIN DOPAMINE SIGNALS

(71) Applicant: SRI International, Menlo Park, CA (US)

(72) Inventors: Christopher Ian Connolly, Meno Park, CA (US); Patrick Denis Lincoln, Menlo Park, CA (US)

(73) Assignee: SRI International, Menlo Park, CA (US)

(21) Appl. No.: 18/810,921

(22) Filed: Aug. 21, 2024

Related U.S. Application Data
(60) Provisional application No. 63/646,062, filed on May 13, 2024.
(60) Provisional application No. 63/646,066, filed on May 13, 2024.

(51) Int. Cl. A61N 1/36; A61N 1/04

(52) U.S. Cl. CPC A61N 1/36025; A61N 1/0456; A61N 1/36031

(57) ABSTRACT

Embodiments are directed to systems, devices, and methods for altering midbrain dopamine signals. An example system comprises stimulation circuitry configured to output a neuromodulation signal to a nerve target of a subject, and processor circuitry configured to cause the stimulation circuitry to output the neuromodulation signal to the nerve target as timed with an event, and in response, cause alteration to midbrain dopamine signals to the subject.

### BACKGROUND

Nerve signals may be stimulated (induced, modified and/or interrupted) using stimulation circuitry. For example, measured peripheral nerve tissue signals can be processed into synthetic neuromodulation signals which can be generated and applied to tissue of a subject for various applications, including but not limited to, therapeutic treatment. A non-limiting example nerve is the vagus nerve, which is located on each side of the human body. The vagus nerve is a component of the autonomic nervous system and plays a role or roles in metabolic and physiologic homeostasis.

Different types of stimulation devices have been used to stimulate nerves or other tissue. Examples include implantable devices that stimulate different tissue for treatment of varying conditions, including heart disease, epilepsy, and depression. These devices are typically implanted through surgery by subcutaneously placing a generator in the upper chest of a patient. An electrode lead is then attached from the generator to the tissue. Other types of devices include transcutaneous stimulation devices. For example, transcutaneous stimulation devices can be used to stimulate the auricular branch of the vagus nerve by targeting the cutaneous receptive field of the auricular branch of the vagus nerve.

### SUMMARY

The present invention is directed to systems, devices, and methods for altering midbrain dopamine signals.

Various embodiments of the present disclosure are directed to a system comprising stimulation circuitry configured to output a neuromodulation signal to a nerve target of a subject, and processor circuitry configured to cause the stimulation circuitry to output the neuromodulation signal to the nerve target as timed with an event, and in response, cause alteration to midbrain dopamine signals to the subject.

In some embodiments, the system further includes memory circuitry in communication with the processor circuitry which stores a depository of a plurality of neuromodulation signals, including the neuromodulation signal, wherein each of the plurality of neuromodulation signals represent a processed nerve tissue signal as a sequence of at least one state corresponding to a set of state parameters and correlated with causing a particular physiological effect. Wherein at least a subset of the plurality of neuromodulation signals, including the neuromodulation signal, are correlated with activating a midbrain dopamine signal pathway to cause alteration to the midbrain dopamine signals.

In some embodiments, the processor circuitry is configured to select the event using a machine learning model that predicts alteration to the midbrain dopamine signals and predicts a condition improvement in response to the output of the neuromodulation signal.

In some embodiments, the processor circuitry includes a machine learning model, which is trained using an input data set including known neuromodulation signals and known effects on the midbrain dopamine signals responsive to the known neuromodulation signals, to identify a transfer pattern that maps the known neuromodulation signals to the known effects.

In some embodiments, the processor circuitry is configured to apply the machine learning model to additional input data to predict a particular neuromodulation signal that is to cause alteration to the midbrain dopamine signals.

In some embodiments, the input data set includes at least one of: applied neuromodulation signals and indication of alteration to the midbrain dopamine signals for the subject; applied neuromodulation signals and indication of alteration to the midbrain dopamine signals for a plurality of other subjects; indication of the applied neuromodulation signals for the subject or for the plurality of other subjects resulting in an intended effect; and timing of the applied neuromodulation signals for the subject or for the plurality of other subjects, and an event.

In some embodiments, the indication of alteration to the midbrain dopamine signals for the subject or plurality of other subjects includes at least one of: a biosignal used as a proxy for dopamine signaling; brain signals indicative of midbrain dopamine spikes; and feedback from the subject.

In some embodiments, the processor circuitry is configured to establish a stimulus program including a sequence of a plurality of additional neuromodulation signals as timed with different events and to cause the stimulation circuitry to output the plurality of additional neuromodulation signals as timed with and in response to the different events to achieve a goal.

In some embodiments, the processor circuitry is configured to cause the stimulation circuitry to output the neuromodulation signal within a threshold time of the event.

In some embodiments, the processor circuitry is configured to cause the stimulation circuitry to output the neuromodulation signal as timed with the event and to cause alteration to the midbrain dopamine signals to cause at least one of: dilution of an addiction cue-related reward, and manipulation of a consumption-related reward or other cue-related reward.

In some embodiments, the processor circuitry is configured to: select at least two stimulation parameters and a plurality of values for the at least two stimulation parameters; cause the stimulation circuitry to output an additional neuromodulation signal to the nerve target which sweeps each of the at least two stimulation parameters to the plurality of values to sample a neuromodulation signal space; determine stimulation parameter ranges for the at least two stimulation parameters that optimize alteration to the midbrain dopamine signals for the subject as a function of the additional neuromodulation signal based on measures of a biosignal received from sensor circuitry responsive to the additional neuromodulation signal; and cause the stimulation circuitry to output the neuromodulation signal that is characterized by the at least two stimulation parameters within the determined stimulation parameter ranges.

In some embodiments, the processor circuitry includes a machine learning model trained to: encode a plurality of measures of a biosignal as pre-images based on respective ones of the plurality of measures of the biosignal obtained without application of neuromodulation signals, wherein the biosignal is associated with the midbrain dopamine signals; and identify a transfer pattern that maps a plurality of additional neuromodulation signals and the plurality of measures of the biosignals using the pre-images and a plurality of additional neuromodulation signals.

Various embodiments of the present disclosure are directed a method comprising determining occurrence of an event associated with a subject, applying a neuromodulation signal to a nerve target of the subject as timed with the event, and causing alteration to midbrain dopamine signals to the subject responsive to the neuromodulation signal applied to the nerve target.

In some embodiments, the method further includes downloading a plurality of neuromodulation signals, including the neuromodulation signal, from external memory circuitry, wherein each of the plurality of neuromodulation signals represent a processed nerve tissue signal as a sequence of at least one state corresponding to a set of state parameters and correlated with causing a particular physiological effect. Wherein at least a subset of the plurality of neuromodulation signals, including the neuromodulation signal, are correlated with activating the midbrain dopamine signal pathway to cause alteration to the midbrain dopamine signals.

In some embodiments, the method further includes selecting the event using a machine learning model which is trained to predict alteration to the midbrain dopamine signals and to predict a condition improvement in response to the neuromodulation signal applied to the nerve target.

In some embodiments, the method further includes identifying a transfer pattern that maps known neuromodulation signals to known effects using a machine learning model which is trained using an input data set including the known neuromodulation signals and the known effects.

In some embodiments, the method further includes using the machine learning model to select the neuromodulation signal from a depository of a plurality of neuromodulation signals based on a prediction that the neuromodulation signal is to cause alteration to the midbrain dopamine signals, wherein each of the plurality of neuromodulation signals represent a processed nerve tissue signal as a sequence of at least one state corresponding to a set of state parameters and correlated with causing a particular physiological effect.

In some embodiments, the method further includes outputting, using the machine learning model, at least one of: a predicted effect of the neuromodulation signal or an additional neuromodulation signal; the event or an additional event to time the neuromodulation signal or an additional neuromodulation signal; and a stimulus program including an additional plurality of neuromodulation signals and events to achieve an effect.

In some embodiments, the method further includes receiving data, from sensor circuitry or other communication circuitry, indicative of the occurrence of the event and, in response, determining the event has occurred and applying the neuromodulation signal within a threshold time of the event occurrence.

Various embodiments of the present disclosure are directed non-transitory computer-readable storage medium comprising instructions that when executed cause processor circuitry to determine occurrence of an event associated with a subject, and cause stimulation circuitry to output a neuromodulation signal to a nerve target of the subject as timed with the event, and in response, cause alteration to midbrain dopamine signals to the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various example embodiments can be more completely understood in consideration of the following detailed description in connection with the accompanying drawings, in which:

FIG. 1 illustrates an example system for stimulating a nerve target to alter midbrain dopamine signals, in accordance with the present disclosure.

FIG. 2 illustrates an example method for altering midbrain dopamine signals, in accordance with the present disclosure.

FIG. 3 illustrates an example computing device including non-transitory computer-readable storage medium storing executable instructions, in accordance with the present disclosure.

FIG. 4 illustrates an example machine learning process, in accordance with the present disclosure.

FIG. 5 illustrates an example system including machine learning models, in accordance with the present disclosure.

FIGS. 6A-6G illustrate example machine learning models, in accordance with the present disclosure.

FIG. 7 illustrates example neurogram recordings, in accordance with the present disclosure.

FIGS. 8A-9 illustrate example results of altering midbrain dopamine signals, in accordance with the present disclosure.

FIGS. 10A-10C illustrate an example of biostimulation signal which sweeps a stimulation parameter and resulting biosignal measures, in accordance with the present disclosure.

FIGS. 11A-11B illustrate example pre-images and cardiac signals output using a machine learning model, in accordance with the present disclosure.

FIGS. 12A-12D illustrate example pre-images, in accordance with the present disclosure.

FIG. 13 illustrates an example of a measured biosignal and a reconstructed biosignal, in accordance with various embodiments.

### DETAILED DESCRIPTION

In the following detailed description, reference is made to the accompanying drawings which form a part hereof, and in which is shown by way of illustration specific examples in which the disclosure can be practiced. It is to be understood that other examples can be utilized, and various changes may be made without departing from the scope of the disclosure. The following detailed description, therefore, is not to be taken in a limiting sense, and the scope of the disclosure is defined by the appended claims. It is to be understood that features of the various examples described herein may be combined, in part or whole, with each other, unless specifically noted otherwise.

Embodiments in accordance with the present disclosure are directed to systems, devices, and/or methods for stimulating a nerve target to alter midbrain dopamine signals to cause a particular effect. The effect can include alleviating addiction, improving learning of a skill, and/or improving athletic performance, among others. In some embodiments, a neuromodulation signal can be output to the nerve target as timed with an event. Midbrain dopamine signals, among other pathways associated with the vagus nerve and/or other nerves, are important for behavioral effects that are controlled by rewards. By timing the application of the neuromodulation signal with the event, reward-related signaling can be manipulated to increase behavior or decrease behavior and/or for other purposes. As the manipulation is reward-related, timing the stimulation to occur within a threshold time of the event can improve the results of the stimulation (e.g., increase likelihood of causing the particular effect).

In some embodiments, a depository of a plurality of neuromodulation signals can be used, which represent processed nerve tissue signals as a sequence of at least one state corresponding to a set of state parameters and causing a particular effect (e.g., physiological, behavioral, and combinations). In some embodiments, the plurality of neuromodulation signals can be implemented to include at least some of substantially the same features and attributes as described by U.S. Pat. No. 11,752,339, issued on Sep. 12, 2023, and entitled “Methods and Systems for Stimulating Nerve Signals”, which is incorporated herein in its entirety for its teaching. For example, applying an electrical signal, as compared to an acoustic signal, to the left and/or right cymba conchae that is above the sensory threshold, but below the pain threshold, can result in brain activation that is similar to that of the left and/or right cervical vagus nerve stimulation.

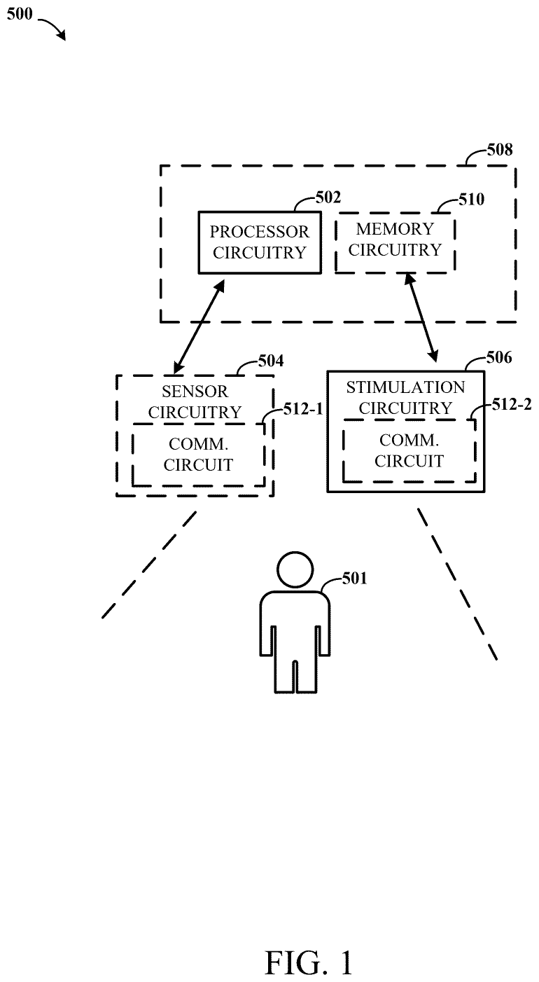

Turning now to the figures, FIG. 1 illustrates an example system for stimulating a nerve target to alter midbrain dopamine signals, in accordance with the present disclosure. The system 500 includes processor circuitry 502 and stimulation circuitry 506.

The stimulation circuitry 506 can output a neuromodulation signal to a nerve target of a subject 501. The stimulation circuitry 506 can include a generator configured to generate neuromodulation signals (e.g., synthetic neuromodulation signals) which emulates stimulation on a target. A neuromodulation signal includes and/or refers to a signal output to stimulate a nerve target of the subject, and which may simulate a neurogram as further described herein. Neuromodulation signals can be waveforms that are delivered with particular stimulation parameters. For example, the generator can deliver the neuromodulation to the nerve target at a particular rate and power.

The stimulation circuitry 506 can use known technologies to apply the neuromodulation signals to the subject 501, including electrical, electromechanical, optical, and acoustic technologies, among others. For example, the stimulation circuitry 506 can include various types of generators, such as but not limited to electrodes, light emitting diodes or other light-emitting devices, mechanical vibrators, radio-frequency transducers, electromagnets, and/or other mechanical or electromechanical components, which can be implemented on various devices, such as speakers, headphones, ear buds, chest straps, smart eye coverings (e.g., glasses, goggles) or virtual reality headsets, among others.

Similarly, the neuromodulation signal (and in some embodiments, a biostimulation signal, as further described herein) can include electrical stimulation signals, acoustic stimulation signals, ultrasound stimulation signals, optical stimulation signals, magnetic stimulation signals, or other types of stimulation and various combinations thereof. In some embodiments, the nerve target is a vagus nerve, such as a right-side vagus nerve. In other embodiments, the nerve target is a left-side vagus nerve, left-side and right-side (e.g., bilateral) vagus nerves, or other nerve targets and different delivery portals. In some embodiments, the target can be associated with or include a delivery portal (e.g., locations). For example, a portion of the ear or ears can be the delivery portal. As other examples, a part of the head, eye(s), and/or chest can be the delivery portal.

As shown, the stimulation circuitry 506 can include communication circuitry 512-2, which provides for communication between the stimulation circuitry 506 and the processor circuitry 502. While not illustrated, the processor circuitry 502 or a computing device 508 that includes the processor circuitry 502 can also include communication circuitry. As further described below, the processor circuitry 502 can communicate with the stimulation circuitry 506 to cause output of neuromodulation signals. In some embodiments, the stimulation circuitry 506 can form part of the computing device 508 with the processor circuitry 502, and in other embodiments, the stimulation circuitry 506 forms part of a device that is separate from the computing device 508 and/or the processor circuitry 502. For example, the stimulation circuitry 506 can form part of a wearable device, such as a headset, headphones, or ear buds, which can be worn in stimulating proximity to the target of the subject 501. In such embodiments, the processor circuitry 502 can form part of the computing device 508, such as a smartphone, tablet, or laptop computer that is in communication with the wearable device.

In some embodiments, the plurality of neuromodulation signals can be stored on a depository, which can be stored on local memory of or associated with the processor circuitry 502 (e.g., memory circuitry 510) or memory circuitry external to, and in communication with, the processor circuitry 502. The depository includes a storage location for plurality of neuromodulation signals (and optionally, other types of biostimulation signals) and can be local to processor circuitry 502 and/or the computing device 508.

In various embodiments, the stimulation circuitry 506 and processor circuitry 502 and/or computing device 508 can be implemented as and/or include at least some of substantially the same features and attributes as the neuromodulation signal generator system and electronic device, including the library storing a set of state parameters for generating synthetic neuromodulation signals, as described by U.S. Pat. No. 11,752,339. In some embodiments, the above-described depository can be formed from example libraries as described by U.S. Pat. No. 11,752,339. The library can include a set a neuromodulation signals and/or other types of biostimulation signals stored in memory circuitry, such as external memory circuitry, that is accessible via a network to form the local depository. A particular subset of the set of neuromodulation signals for use in a particular application can be obtained (e.g., downloaded) by the processor circuitry 502. For example, the plurality of neuromodulation signals can be downloaded from the library to form a local depository as stored on the memory circuitry 510 and which are associated with the specific effect on the subject 501. Other effects can be identified and, in response, additional neuromodulation signals (and/or other types of biostimulation signals) can be downloaded and stored in the depository and applied to the subject 501. The library can be accessed through any suitable network or communications link, including wireless, optical or wired computing systems. Each of the neuromodulation signals on the library can describe at least one state along with control data that contains information about how the neuromodulation signals can be used. In some embodiments, the processor circuitry 502 can access digital representations of the neuromodulation signals from the library and output the digital representations of the neuromodulation signals to the stimulation circuitry 506 and the stimulation circuitry 506 can convert the digital representations to the analog domain for application by the generator.

In some embodiments, the plurality of neuromodulation signals can include electrical stimulation signals, acoustic stimulation signals, ultrasound stimulation signals, optical stimulation signals, magnetic stimulation signals, or other types of stimulation and various combinations thereof. The stimulation circuitry 506 can generate and output the plurality of neuromodulation signals to the nerve target of the subject 501. In some embodiments, the nerve target is a peripheral nerve, such as the vagus nerve. However, embodiments are not so limited and other locations or nerve targets can be used, as further described herein.

In some embodiments, each of the plurality of neuromodulation signals can represent at least one processed measured nerve tissue signal as a sequence of at least one state represented by at least one state parameter. The state parameters can define the waveform of the measured nerve tissue signal, such as including waveform parameters, amplitude mean and variance, firing rate mean and variance. As further described herein, state parameters defining the measured nerve tissue signal waveform can be adjusted to account for the neuromodulation signal transforming when penetrating through skin and other tissue to the nerve target and/or can define or include the stimulation parameters. Said differently, the state can describe a neuromodulation signal or a recorded neurogram. For neuromodulation, the sequence of states is for a desired effect, with state parameters assigned to each state. For a recorded neurogram, the neurogram is captured and translated to the sequence of states represented by the state parameters that fit the recording.

Stimulation of nerve tissue can be based on neuromodulation signals generated based on stimulation parameters. Stimulation parameters include and/or refer to parameters that define the waveform of the neuromodulation signals. Example stimulation parameters include pulse frequency, duration, amplitude, duty cycle, pulse width, delivery portal, and a combination thereof. In some embodiments, stimulation of the target nerve can be based on newly defined neuromodulation signals that are determined to have a beneficial effect on the subject 501. The systems, methods, and devices disclosed herein can enable generation of these stimulus patterns without requiring surgery, or prior recordings of nerve or other tissue functions. The neuromodulation signals can be presented to an individual through a variety of means. For example, the neuromodulation signals can be presented through sound vibrations, light stimulation, and other devices attached to the ear or eye that are configured to stimulate nerves, such as the vagus nerve.

Embodiments disclosed herein can provide a convenient, safe, and effective way for the development and use of neuromodulation techniques. In some embodiments, the technologies described herein can provide personalized health and/or behavioral benefits. In some embodiments, subjects can be able to directly manage their individual health condition through an automated system, which can include a user-friendly human-computer interface, instructions implemented in software, and hardware including processor(s), memory, and input/output device(s). As previously described, some embodiments include a library and/or depository of neuromodulation signals, such as synthetic neuromodulation signals. Each neuromodulation signal can correspond to a specific pattern that has been correlated with a particular desired effect. For example, stored synthetic neuromodulation signals for generating a neuromodulation signal (NMS) #1 can be useful to treat depression. A subject or other person (e.g., a user) can download the synthetic neuromodulation signal from the library, and load it into a computing device 508 or directly onto stimulation circuitry 506 (on the electronic device or stand-alone) configured to stimulate tissue. By playing the NMS #1 on his or her device, the subject 101 can be treated for a disorder, addiction, and/or other purposes without the need to resort to pharmaceutical medications. The depository can include neuromodulation signals for a variety of effects (such as alleviating addiction, treating a disorder, learning a skill, learning or improving an athletic skill or performance), as further described below. The effects can be associated with stimulations of the vagus nerve, or other nerves and/or tissue in the body.

In some embodiments, the neuromodulation signals can be generated by measuring a peripheral nerve tissue signal taken from a subject subjected to a condition; creating a synthetic neuromodulation signal by representing at least one of the measured peripheral nerve tissue signals (e.g., neurograms) as a sequence of at least one state, wherein each state is represented by at least one state parameter that is/are converted to the synthetic neuromodulation signal; and sending the synthetic neuromodulation signal to the stimulation circuitry 506 configured to apply the synthetic neuromodulation signal to the subject 501, wherein application of the synthetic neuromodulation signal to the subject 501 causes the subject 501 to experience an intended effect and which may be without application of the condition to the subject 501.

As further described herein, embodiments are not limited to neuromodulation signals, and the above can be applied to other types of biostimulation. In any of the above and below described embodiments, the term “neuromodulation signal” can be replaced with “biostimulation signal”. A biostimulation signal includes and/or refers to a signal output to stimulate the target of the subject, and can include non-neural signals and/or neuromodulation. For example, a tissue signal (e.g., brain signal, acoustic signal, electromyogram, or other type of signal) can be recorded from a subject subjected to a condition; following, a synthetic biostimulation signal can be created by representing at least one of the measured signals as a sequence of at least one state, with each state represented by state parameter(s). As a specific example, for acoustic biostimulation, the state parameters can be similar to those for electrical stimulation. For stimulating organs, ultrasound can be used to modulate the behavior of non-neural tissue to achieve the effects in non-neural biosignals (e.g., cytokines). In some such embodiments, a mode of biostimulation can be defined that specifies the kind of energy to be used for biostimulation (e.g., electrical, acoustic, ultrasound) and the target where the energy is to be applied (e.g., cymba, concha, spleen). In some embodiments, the biostimulation can be multi-modal stimulation that is applied in parallel, where the states can specify the mode (e.g., ultrasound verses electrical). In such embodiments, the state machine can diverge (e.g., fork) and transition to two or more states in parallel, one for each mode. The state path(s) can diverge and then join, with divergence and joining indicating when states are executed in parallel.

The processor circuitry 502 can cause the stimulation circuitry 506 to output the neuromodulation signal to the nerve target as timed with an event, and in response, cause alteration to midbrain dopamine signals to the subject 501. In some embodiments, the output of neuromodulation signal can impact other neurotransmitters in addition or alternative to dopamine, such as norepinephrine, serotonin, and acetylcholine using a similar pathway and timing. The neuromodulation signal can be timed to be output within a threshold time of the event, such as within a sub-second range. Timing the neuromodulation signal with the event can cause a particular intended effect.

As noted above, midbrain dopamine signals are important for behavioral effects that are controlled by rewards. As may be appreciated, there are two-component phasic dopamine responses including an initial activation (e.g., novelty signal or reward-related signal) and a subsequent activation or depression code that encodes a positive or negative value signal(s) (e.g., value signal). More particularly, the midbrain dopamine signals can include a value prediction signal which is indicative of a predicted value of an event (e.g., which can be associated with a cue), an obtained value signal which is indicative of the actual value of the event, and a reward prediction error (RPE) signal that shows the difference between the value prediction signal and obtained value signal, e.g., difference between the reward expected and the reward received.

In various embodiments, the event that the neuromodulation is timed to can include a physiological event and/or a behavioral event. Example physiological events include a respiratory event, a cardiac event, and physiological threshold associated thereof, which may be determined based on measures of biosignals. The behavioral event can be an endogenous behavioral event (e.g., self-initiated) or an exogenous behavioral event (e.g., external cues). Example endogenous behavioral events include consumption of food or liquid, movement or other motor actions (e.g., movement of the arms or limbs, grasping, exercise), learning a skill or improving on a skill, among others. Example exogenous behavioral event include external cues, such as environmental stimuli including visual, auditory, and other cures. A behavioral event can be measured using signals and/or data other than biosignals. In some embodiments, the event is a non-respiratory event.

The event can be associated with a goal for stimulation. For example, the neuromodulation signal can be applied to reduce addictive behavior, to treat a disorder or physiological issue, to learn a skill or improve performance, such as for athletics, among other effects and combinations thereof. As further described herein, the processor circuitry 502 can identify occurrence of the event, such as via intrinsic or extrinsic data sources, and in response, cause the stimulation circuitry 506 to output the neuromodulation signal.

The effect(s) can include a physiological effect and/or a biological effect. As used herein, a physiological effect includes and/or refers to a change or response in the body which can be measured, such as via biosignals or environmental state signals. A behavioral effect includes and/or refers to a change or response to behavior of the subject, and which may not be measured or may be difficult to measure directly. Example physiological effects include changes in biosignal measures or maintaining particular values, altering midbrain dopamine signals, among others. Example behavioral effects include learning or improving a skill, improving athletic performance, reduction in addictive behaviors or other habits, among others. In some embodiments, the effects can include combinations of physiological effects and behavioral effects, such as for treatment of a disorder.

In some embodiments, the system 500 further includes memory circuitry 510 in communication with the processor circuitry 502. The memory circuitry 510 can store a depository of a plurality of neuromodulation signals (and optionally other biostimulation signals) including the neuromodulation signal output to the subject 510. Each of the plurality of neuromodulation signals can represent a processed nerve tissue signal (e.g., a neurogram) as a sequence of at least one state corresponding to a set of state parameters and correlated with causing a particular physiological effect, as previously described. In some embodiments, at least a subset of the plurality of neuromodulation signals, including the neuromodulation signal, are correlated with activating the midbrain dopamine signal pathway to cause alteration to the midbrain dopamine signals.

In some embodiments, as noted above, the processor circuitry 502 can form part of a computing device 508. The computing device 508 can further include the memory circuitry 510 that stores instructions executable by the processor circuitry 502. In other embodiments, the processor circuitry 502 and/or the memory circuitry 510 can form a part of distributed computing devices, with the distributed computing devices being in communication with each other.

In some embodiments, the processor circuitry 502 can cause the stimulation circuitry 506 to output the neuromodulation signal as timed with the event and to cause alteration to the midbrain dopamine signals to cause at least one of: (i) dilution of an addiction cue-related reward; and (ii) manipulation of a consumption-related reward or other cue-related reward. The addiction cue-related reward can include or be associated with an RPE signal which is responsive to an addiction cue (e.g., an event associated with addictive behavior). The consumption-related reward can include or be associated with an RPE signal which is responsive to consuming liquid or food, which may be used to mitigate addictive behaviors, as further described below. Other cue-related reward can include or are associated with an RPE which is responsive to other types of cues, such as events associated with learning or improving a skill.

In some embodiments, the event is associated with addiction, as further described herein. In some such embodiments, the alteration to the midbrain dopamine signals can cause dilution of an addiction cue-related reward or manipulation of a consumption-related reward in the subject 501.

In some embodiments, the event is associated with learning a skill or achieving goal, such as for athletics as further described herein. In some such embodiments, the alteration to the midbrain dopamine signal can cause manipulation of a cue-related reward in the subject 501.

In some embodiments, the processor circuitry 502 includes or accesses a machine learning model, which is trained using an input data set to identify a transfer pattern that maps the known neuromodulation signals to the known effects. The machine learning model can be trained using an input data set including the known neuromodulation signals and known effects on the midbrain dopamine signals responsive to the known neuromodulation signal. In some embodiments, the input data set further includes known events and timings of the known neuromodulation signals. In some embodiments, as further described below, the system 500 can output an indication of a predicted effect of a neuromodulation signal, an additional neuromodulation signal to output, an event to time the additional neuromodulation signal to, and/or a stimulus program predicted to cause an intended effect including additional neuromodulation signals to time with specific events, as further described herein. In some embodiments, the system 500 can cause output of an additional neuromodulation signal to the target of the subject 501.

In various embodiments, the input data set can include at least one of: (i) applied neuromodulation signals and indication of alteration to the midbrain dopamine signals for the subject 501; (ii) applied neuromodulation signals and indication of alteration to the midbrain dopamine signals for a plurality of other subjects; (iii) indication of the applied neuromodulation signals for the subject 501 or for the plurality of other subjects resulting in an intended effect (e.g., learning, improvement in addiction, improved athletic performance); and (iv) timing of the applied neuromodulation signals for the subject 501 or for the plurality of other subjects and an event. In some embodiments, indications of alteration to the midbrain dopamine signals for the subject 501 or plurality of other subjects include at least one of a biosignal used as a proxy for midbrain dopamine signaling, brain signals indicative of midbrain dopamine spikes, and feedback from the subject 501. For example, and as described above, a cardiac signal can be used as a proxy for brain signals. In some embodiments, the indications of the applied neuromodulation signals for the subject 501 or for the plurality of other subjects resulting in the intended effect can be obtained from feedback from the subject 501 (e.g., biosignals, environmental sensor signals, input manually by the subject 501), from a plurality of other subjects, or from another source confirming the intended effect (e.g., a coach or health professional).

In some embodiments, the processor circuitry 502 selects the event using a machine learning model that predicts alteration to the midbrain dopamine signals and/or predicts a condition improvement in response to the output of the neuromodulation signal, such as for health or disorder treatment, learning, addiction, and/or athletics. For example, based on the input data set, the machine learning model can predict application of a neuromodulation signal timed with an event will cause an intended effect, such as those described above. The condition improvement can include physiological and/or behavioral effects. For some condition improvements, such as with improving addiction, the prediction can include a series of events and neurostimulations (e.g., a stimulus program).

In some embodiments, the processor circuitry 502 applies the machine learning model to additional input data to predict a particular neuromodulation signal that is to cause alteration to the midbrain dopamine signals. As previously described, the additional input data can include known neuromodulation signals and effects on the midbrain dopamine signals which can be obtained from data obtained from a plurality of other subjects (e.g., demographic data) and/or data obtained from the subject 501.

In some embodiments, the system 500 further includes sensor circuitry 504. The sensor circuitry 504 can obtain measures of a biosignal from the subject 501. Example biosignals include cardiac signals (e.g., heart rate (HR), heart rate variability (HRV), electrocardiograph (ECG)), body or skin temperature data, respiratory data (e.g., respiratory rate, tidal volume), oxygen levels or saturation (e.g., photoplethysmograph (PPG)), blood pressure, brain, muscle, and/or or nerve data (e.g., electromyogram (EMG), electroencephalogram (EEG), electroneurogram (ENG), neural responses which are captured using magnetic resonance imaging (MRI)), skin conductance or galvanic response, hormone levels, among others measures as well as combinations thereof. A biosignal includes and/or refers to a signal measured from a living thing. In some embodiments, the sensor circuitry 504 can collect additional signals. For example, the sensor circuitry 504 can collect biosignals that are continuous and discrete and which are derived from a biological state (e.g., intrinsic) and/or signals derived from an environmental state (e.g., extrinsic). Example signals derived from an environmental state include acoustic signals, temperature, movement signals (e.g., motion, steps), and optic signals, among other signals.

In some embodiments, the sensor circuitry 504 can measure data indicative of occurrence of the event. For example, the sensor circuitry 504 can include a motion sensor, light sensor, sensor to detect consumption or swallowing, a camera or other optical sensor for object recognition, a biosensor, among other types of sensors. In some embodiments, the sensor circuitry 504 can collect signals that are continuous and discrete and which are derived from the biological state (e.g., intrinsic) and/or derived from the environmental state (e.g., extrinsic), as previously described.

In some embodiments, the sensor circuitry 504 can include wearable technology, such as wearable devices, sensors and/or environmental sensors, among others. Wearable technology, as used herein, includes and/or refers to one or more sensors and/or device with at least one sensor that is wearable and used to obtain a biosignal (e.g., physiological) measurements from the subject 501 or extrinsic measurements derived from the environment. In some embodiments, the wearable technology can be continuously worn by the subject 501 for a period of time or periods of time (e.g., for a day, for multiple days, for months, all day, all night). Non-limiting examples of wearable technology include a smart watch, fitness watch, smart ring, a chest strap, a headset, a headphone or ear bud(s), among others. In some embodiments, alternatively or additionally, the sensor circuitry 504 can include non-wearable sensors and/or other technology, which can be embedded in a particular location and/or environment. Similar to the stimulation circuitry 506, the sensor circuitry 504 can include communication circuitry 512-1, which provides for communication between the sensor circuitry 504 and the processor circuitry 502.

In some embodiments, the system 500 can further include communication circuitry 512-1, 512-2, which allow for communication to the processor circuitry 502. As previously described, the sensor circuitry 504 and/or stimulation circuitry 506 can include communication circuitry 512-1, 512-2 to communicate data to the processor circuitry 502. For example, communication circuitry 512-1 can receive input indicative of occurrence of the event and communicate with the processor circuitry 502. While communication circuitry 512-1 is shown as part of the sensor circuitry 504, in some embodiments, the communication circuitry 512-1 can be separate therefrom and form part of another device for the subject 501 or other person to input data indicative of the occurrence of the event and communicate with the processor circuitry 502.

In some embodiments, as further described herein, the processor circuitry 502 can select at least two stimulation parameters and a plurality of values for the at least two stimulation parameters, and cause the stimulation circuitry 506 to: (i) output an additional neuromodulation signal to the nerve target which sweeps each of the at least two stimulation parameters to the plurality of (different) values to sample a neuromodulation signal space, (ii) determine stimulation parameter ranges for the at least two stimulation parameters that optimize alteration to the midbrain dopamine signals for the subject 501 as a function of the additional neuromodulation signal based on measures of a biosignal received from sensor circuitry 504 responsive to the additional neuromodulation signal, and (iii) cause the stimulation circuitry 506 to output the neuromodulation signal that is characterized by the at least two stimulation parameters within the determined stimulation parameter ranges. The above can be referred to as a process to expand the stimulation space, which can be applied independent of the event timing stimulation. Embodiments are not so limited, and in some instances, a single parameter may be swept to a plurality of values. In such embodiments, the neuromodulation signal output to the target can include different values for the stimulation parameter. Example stimulation parameters that can be swept include pulse frequency, duration, amplitude, duty cycle, pulse width, delivery portal, and a combination thereof.

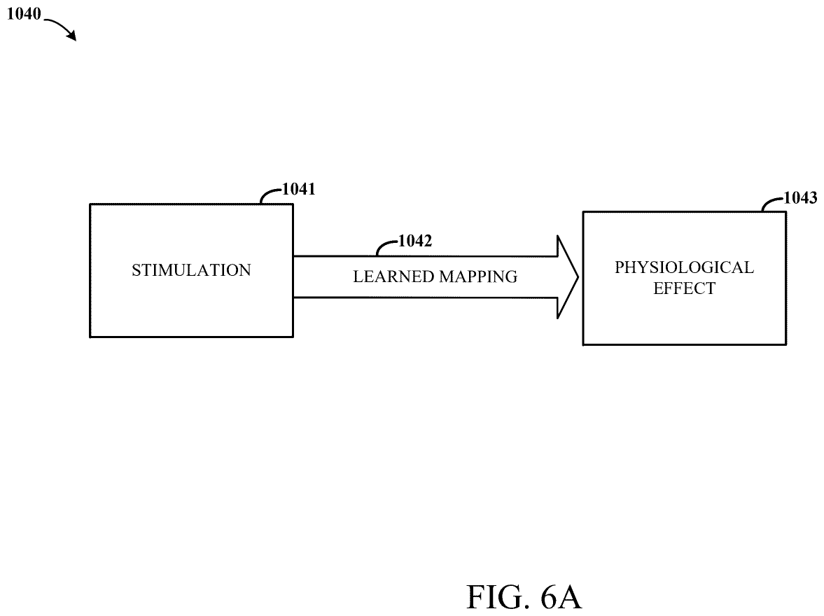

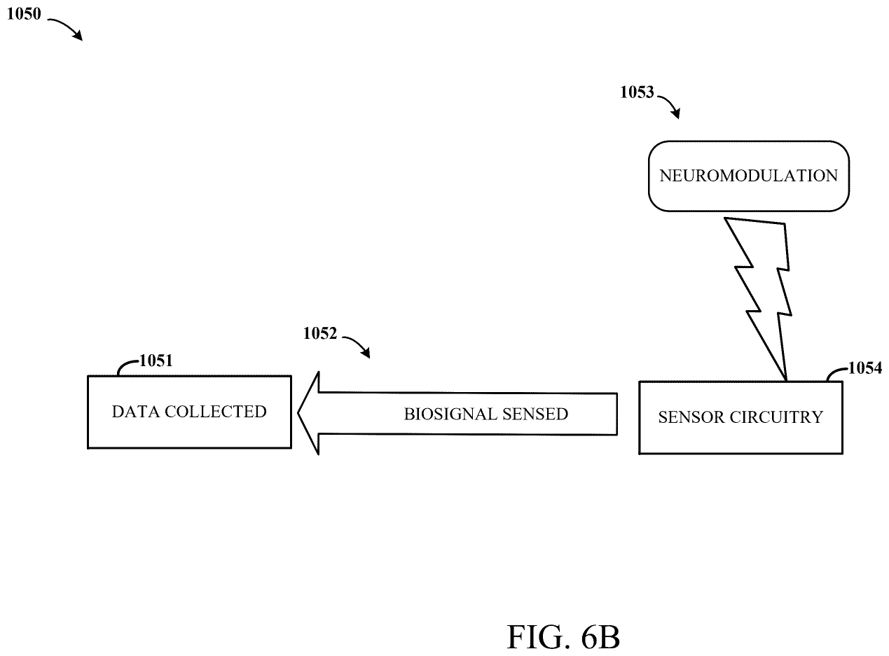

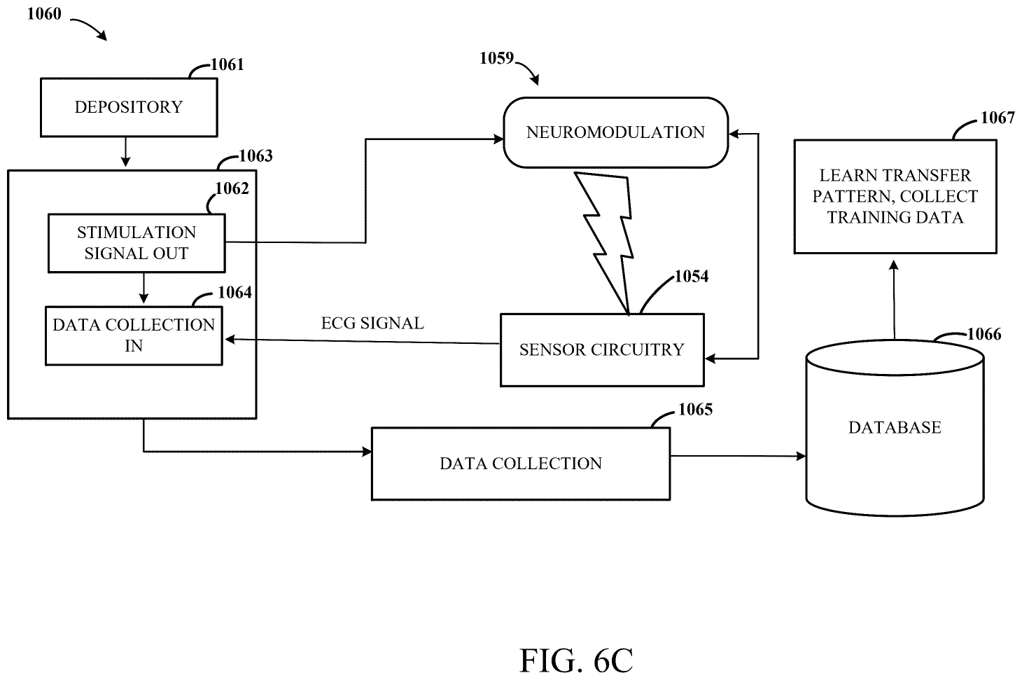

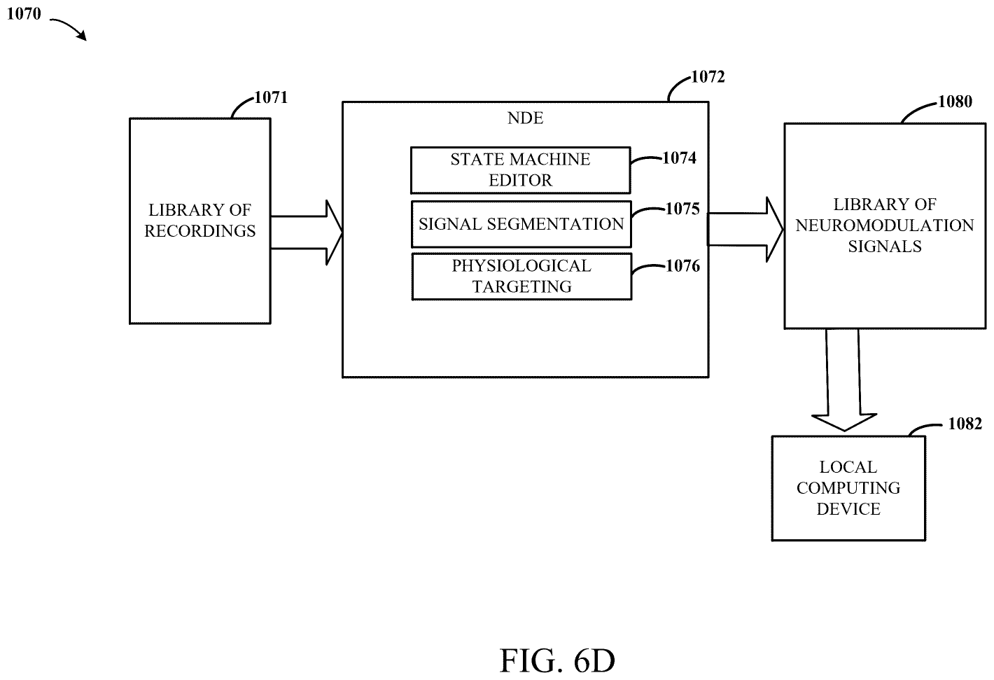

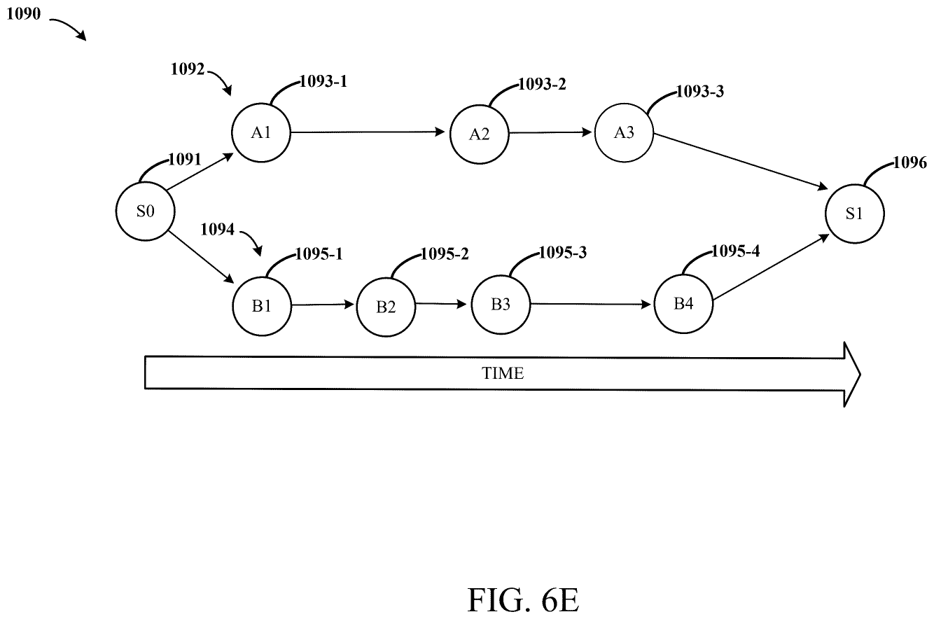

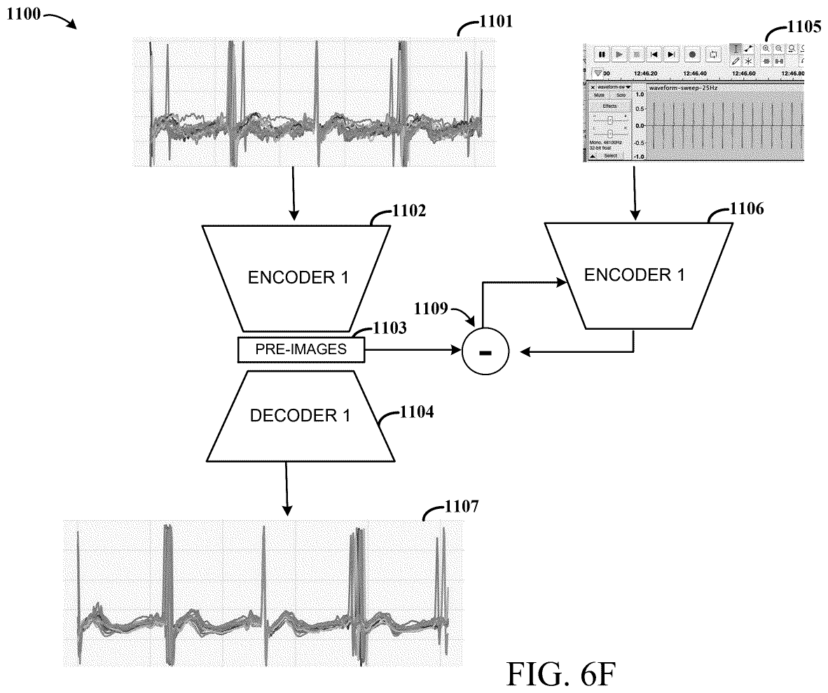

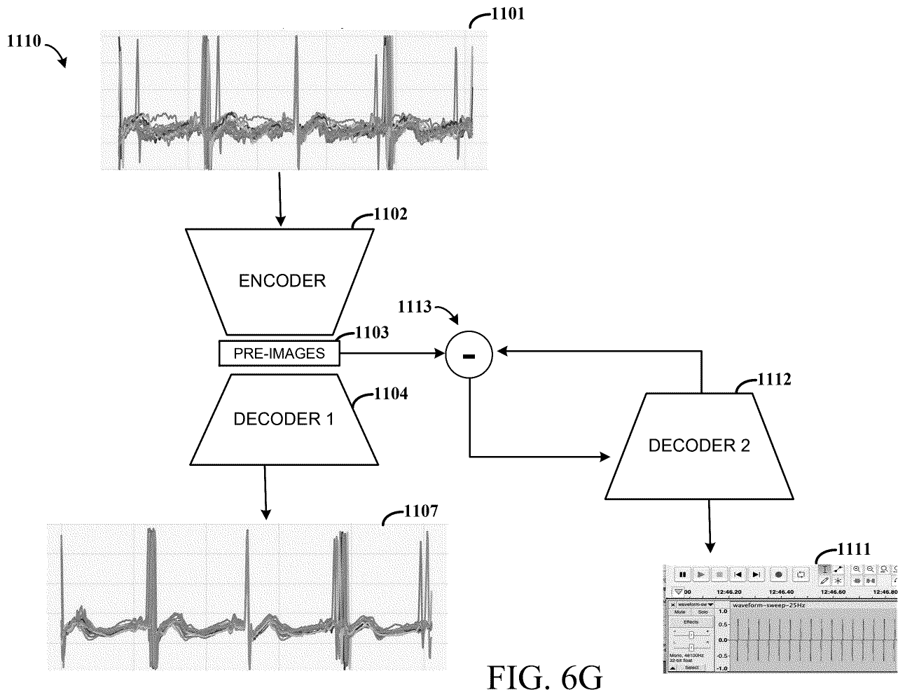

As noted above, the processor circuitry 502 can include or have access to a machine learning model which is trained using known inputs and known outputs. In some embodiments, the machine learning model can be trained to: (i) encode a plurality of measures of a biosignal as first pre-images based on respective ones of the plurality of measures of the biosignal obtained without application of neuromodulation signals, wherein the biosignal is associated with the midbrain dopamine signals, and (ii) identify a transfer pattern that maps a plurality of additional neuromodulation signals and the plurality of measures of the biosignals using the first pre-images and the plurality of additional neuromodulation signals. Pre-images include and/or refer to compressed versions of the input (e.g., biosignal waveform), such as a low dimensional hidden state including reduced features as compared to the input waveform. As further illustrated and described in connection with FIGS. 6F-6G, the machine learning model can include a modified autoencoder, which can be applied independent of the timing stimulation to an event. For example, the modified autoencoder can include: (i) an encoder, and (ii) a second encoder and a decoder or two decoders. In some embodiments, the modified autoencoder can include at least two machine learning models including an autoencoder (e.g., an encoder and decoder) and a second encoder or second decoder. The processor circuitry 502 can use the autoencoder output as an input to the second encoder or second decoder. In some embodiments, the transfer pattern can be used by the system 500 to output an indication of or cause the output of the plurality of additional neuromodulation signals to the nerve target of the subject 501 to cause a particular effect.

In various embodiments, the processor circuitry 502 can establish a stimulus program that causes the stimulation circuitry 506 to output a plurality of additional neuromodulation signals (and/or other types of biostimulation signals) to the subject 501 or other subjects and timing for the additional neuromodulation signals to achieve a goal (e.g., condition improvement). For example, the additional neuromodulation signals can be timed with and in response to different events. The goal can include a physiological effect (e.g., biosignal measure, impact dopamine signals, disorder symptom improvement) and/or a behavioral effect (e.g., improve learning and/or treat addiction), as described above. For example, the processor circuitry 502 can provide the quantified responsiveness as an input to a machine learning model to predict a stimulus program for the subject 501 or another subject that achieves a goal using the learned transfer pattern. The stimulus program can include a sequence of a plurality of (additional) neuromodulation signals and timed with different events, and which can be guided by feedback from the sensor circuitry 504, an extrinsic data source, or a combination thereof.

In some embodiments, the biosignal can be used as a proxy for a second biosignal. In such embodiments, the processor circuitry 502 can include or have access to a machine learning model which is trained to: (i) identify a first transfer pattern that maps the measures of the second biosignal and the plurality of neuromodulation signals; and (ii) identify a second transfer pattern that maps the measures of the biosignal and the second biosignal. The processor circuitry 502 can input the biosignal, as a proxy for the second biosignal, to the machine learning model or a second machine learning model and to predict an effect of an additional neuromodulation signal on the second biosignal. In some embodiments, the system 500 can further include second sensor circuitry configured to obtain measures of a second biosignal from the subject 501. The biosignal may be easier to capture using wearable or other technology than the second biosignal.

As described above, various embodiments involve the use and/or training of a machine learning model. Any of the above and below-described machine learning models can be trained by the processor circuitry 502 or other circuitry using an input dataset of known inputs and known outputs to identify a transfer pattern, among other applications.

In some embodiments, the processor circuitry 502 can train the machine learning model based on general population trends and demographic information associated with the subject 501. For example, the machine learning model can be trained using known inputs, such as demographically similar subjects (and/or the subject), and with the known outputs. Example known inputs include neuromodulation signals including stimulation parameter(s), events, timing of the events and/or neuromodulation signal output, among other data. Example known outputs include biosignal measures or other physiological effects and/or behavioral effects. The input data used to train the machine learning model can include intrinsic and extrinsic data sources.

In some embodiments, the machine learning model is initially trained using demographic data and/or data of other subjects such that the machine learning model can be referred to as a demographic-machine learning model, which may not be specific to the subject 501 and/or may be a function of the particular demographic (e.g., age, sex, race, hereditary information). The demographic-machine learning model can be based on average trends for subjects of the particular demographic. The processor circuitry 502 can revise the demographic-machine learning model to be specific to the subject 501 to generate a subject-specific machine learning model based on data specific to the subject 501. This can include retraining or revising the machine learning model (e.g., the demographic machine learning model) using the subject-specific data.

Example demographic patterns for a subject can be based on age, sex, race, and/or other information, although embodiments are not so limited. In some embodiments, further demographic patterns can be learned by machine learning model over time, such as based on biosignals from a plurality of subjects.

As used herein, machine learning models can include data models which estimate or provide an output based on input data. Various machine learning frameworks are available from multiple providers which provide open-source machine learning datasets and tools to enable developers to design, train, validate, and deploy machine learning models, such as machine learning processors. Machine learning processors (sometimes referred to as hardware accelerators (MLAs), or Neural Processing Units (NPUs)) can accelerate processing of machine learning models. Machine learning processors are integrated circuits (ASICs) that can have multi-core designs and employ precision processing with optimized dataflow architectures and memory use to accelerate calculation and increase computational throughput when processing machine learning models.

Example machine learning models include artificial neural network, support vector machine (SVM), deep learning, cluster, and/or other models. An artificial neural network can estimate a function(s) that depends on inputs. In some embodiments, one or more layers of artificial neurons can receive input data and generate output data. Neural networks can include networks such as, but not limited to, learning networks (e.g., deep, deep structured, hierarchical, and the like), convolutional, auto-type networks (e.g., autoencoder, auto-associator), Diablo networks, and neural network models (e.g., feedforward, recurrent).

An SVM can utilize a linear classification. This classification can act to separate the data points into classes based on distance of the data points from a hyperplane. In some embodiments, the hyperplane is arranged to maximize the distances from the hyperplane to the nearest data points on either side of the hyperplane. This arrangement can group points located on opposite sides of the hyperplane into different classes. However, in some embodiments, the SVM can include a nonlinear classification that separates the data points with a hyperplane in a transformed feature space. The transformed feature space can be determined by one or more kernel functions, including nonlinear kernel functions. In some embodiments, the SVM is a multiclass SVM that separates data points into more than two classes, which can reduce a multiclass problem into multiple binary classification problems.

In some embodiments, a deep learning model can include models such as, but not limited to, convolutional networks (e.g., deep belief, neural), belief networks, Boltzmann machines, deep coding networks, stacked autoencoders, stacking networks (e.g., deep or tensor deep), hierarchical-deep models, deep kernel machines, and the like. It will be understood that such embodiments can include variants and/or combinations of the above-noted example networks.

In some embodiments, the machine learning model(s) can include a clustering method(s), which can include hierarchical clustering, k-means clustering, density-based clustering, among others. In some embodiments, the hierarchical clustering can be used to construct a hierarchy of clusters of the set of features. In some embodiments, the hierarchical clustering utilizes a “bottom up” approach (e.g., agglomerative) wherein each data point starts in its own cluster, and pairs of clusters are merged at progressively higher levels of the hierarchy. However, in some embodiments, the hierarchical clustering utilizes a top-down approach in which all data points start in one cluster, and then clusters are split at progressively lower levels of the hierarchy.

In some embodiments, the k-means clustering implementation can include placing the set of features into k clusters, where k is an integer equal or greater than two. Via such clustering, each data point belongs to a cluster having a mean that is closer to the data point than any means of the other clusters. However, in some embodiments, a machine learning model can include density-based clustering, which can be used to group together data points that are close to one another, while identifying as outliers any data points that are far away from other data points.

In some embodiments, a machine learning model can include a mean-shift analysis that can be used to determine the maxima of a density function based on discrete data sampled from that function.

In some embodiments, a machine learning model can include structured prediction techniques and/or structured learning techniques. Such techniques can be used to predict structured objects and/or structured data, such as structured sets of features and/or sensor data. In some embodiments, such structured prediction and/or structured learning techniques can include graphical models, probabilistic graphical models, sequence labeling, conditional random fields, parsing, collective classification, bipartite matching, Bayesian networks or models, and the like. It will be understood that such embodiments include variants and/or combinations of the above-noted example techniques.

Machine learning can be useful as biosystems may not be linear. Stimulation must penetrate tissue and the neuromodulation waveform at the target may not be the waveform emitted by the stimulation circuitry 506 (e.g., an electrode). For example, the waveform emitted by the stimulation circuitry 506 can be transformed when penetrating through tissue to the nerve target of the subject 501, which is complex. Further, various physiological effects, such as activation of brain patterns, can be difficult to measure directly and can be linked to a second biosignal that is easier to sense. Similarly, the relationship between neuromodulation signals (e.g., waveforms, modality, timing) and the effect can be identified by the machine learning model, which are complex relations. Machine learning can enable a control loop via use of neuromodulation signals stored for evoking physiological and/or behavioral effects from sequences of stimulation. The effects can be combined to impact multiple effects, such as using taVNS to control seizures for an epileptic subject and also enhance a learning skill or other behavior.

In some specific embodiments, the system 500 can be used to alter midbrain dopamine signals of the subject 501 for alleviating addiction through ta VNS. Many addictive behaviors arise in response to midbrain dopamine signals, which at least partially encode for cue-related reward predictions. The application of taVNS as applied to the right-side vagus nerve can dilute or reduce addiction cue-related reward and/or reduce or otherwise manipulate a consumption-related reward, which respectively are related to the RPE signal.

Some anti-addiction strategies rely on pharmaceutical or incentive-based approaches. Pharmaceutical approaches can come with other risks due to broad effects, such as application to the nervous system. Incentive-based approaches, while effective, are best at indirect attempts to manipulate the dopamine RPE signal by using a proxy for dopamine (e.g., money) and is entirely based on the subject's perception of reward. In contrast, embodiments of the present disclosure manipulate the RPE-related dopamine signals in a measurable way via right-side VNS and as customized via events which are relevant cues and responses to addictive behavior.

When habits are learned, particularly for addiction, relevant cues in the environment can give rise to a prolonged increase in dopamine firing whose magnitude is associated with the expectation of reward, and which can be referred to as the value prediction signal. Upon completion of a habitual task (e.g., drinking, drug use), the dopamine signal indicates the prediction error (e.g., “was this better or worse than I expected?”), which can be referred to as the RPE signal. The timing of these signals is important, with the precision being in the sub-second range, e.g., some hundreds of milliseconds after the appearance of the event, such as a cue or consumption.

Systems, devices, and methods as described herein can create tuned VNS programs that are designed to reduce the association of the cue and reward by: (i) inducing dopamine spikes or ramps correlated with irrelevant cues (thus decreasing the salience of the original cue) (e.g., dilute the value prediction signal); and/or (ii) manipulating the reward prediction error (e.g., the RPE signal) so that either (a) a different substance (e.g., water) evokes a strong positive RPE (e.g., positive obtained value signal and/or RPE signal for another substance that is positive behavior) or (b) the addictive substance evokes a strong negative prediction error (e.g., negative obtained value signal and/or RPE signal for addictive substance).

The systems, devices, and/or methods described herein can be agnostic about the delivery portal of stimulation. For example, the stimulation can be cervical or auricular VNS. The neuromodulation signal can be titrated to customize and optimize the effect of the VNS programs. Examples include addiction to social media, where minimal instrumentation is necessary, and alcohol or other substance addiction. For substance addiction, gaze tracking in conjunction with object detection and recognition can be useful for identifying cues and customizing the neuromodulation signal based on the attention of the subject to an object, such as a bottle or drug paraphernalia. Other sensors and/or instrumentation can allow for detection of swallowing events and to provide right-side VNS to manipulate RPE after ingestions.

In some embodiments, the systems, devices, and methods can involve creating VNS patterns for use with the right-side vagus nerve. The signal patterns are designed to alter midbrain dopamine signals to extinguish the additive behavior. The neuromodulation signal can be applied to afferent fibers of the vagus nerve to cause simulation of the dopamine signals, such as those noted above.

Embodiments are not limited to treating addiction, and can be directed to a variety applications, including different disorder treatments, skill learning, and athletic performance improvement. Additionally, different delivery portals can be used, including left-side VNS, bilateral VNS, and combinations of left-side, right-side, and bilateral VNS, as well as different targets and locations other than the ear.

Motor skill learning for learning a particular skill, such as in sports, can be dependent on dopamine. Various embodiments are directed use of timed dopamine stimulation via VNS to improve learning. Such improvement in learning can be used for treatment of a disorder (e.g., Parkinson's disease, attention disorders), improve learning, and/or for athletic purposes, among others.

In some embodiments, the neuromodulation can be timed with an athletic-related event to boost the RPE signal. The neuromodulation signal can be applied as timed with occurrence of the athletic-related event to improve athletic performance in single player and multi-player sports, among other purposes. The neuromodulation signal can be applied via taVNS to elicit midbrain dopamine spikes and/or ramps, such as via right-side ta VNS. The neuromodulation signal can be benign, transcutaneous, and non-invasive. The neuromodulation signal can boost the dopamine signal (e.g., obtained value signal and/or RPE signal) when a motor skill (or other learned skill) is executed.

For multi-players sports, such as team sports, multiple players can be stimulated in coordination to improve group performance. In some such embodiments, the stimulation circuitry 506 can include a plurality of stimulation circuits, each being associated with a different subject of an athletic team. The processor circuitry 502 can cause each of the plurality of stimulation circuits to output the neuromodulation signal as timed with the event to each of the plurality of subjects, such that the plurality of subjects are stimulated in coordination (e.g., at the same time or near same time). In some such embodiments, the system 500 further includes the sensor circuitry 504 to measure data indicative of occurrence of the event, such as a sensor to detect a strike zone, a goal, or other athletic-related event, radio frequency identification (RFID) tags on the equipment or other sensors. The system 500 can alternatively or further include communication circuitry 512-1 to receive input of occurrence of the event from the sensor circuitry 504 or as input by a person and communicate with the processor circuitry 502, as previously described.

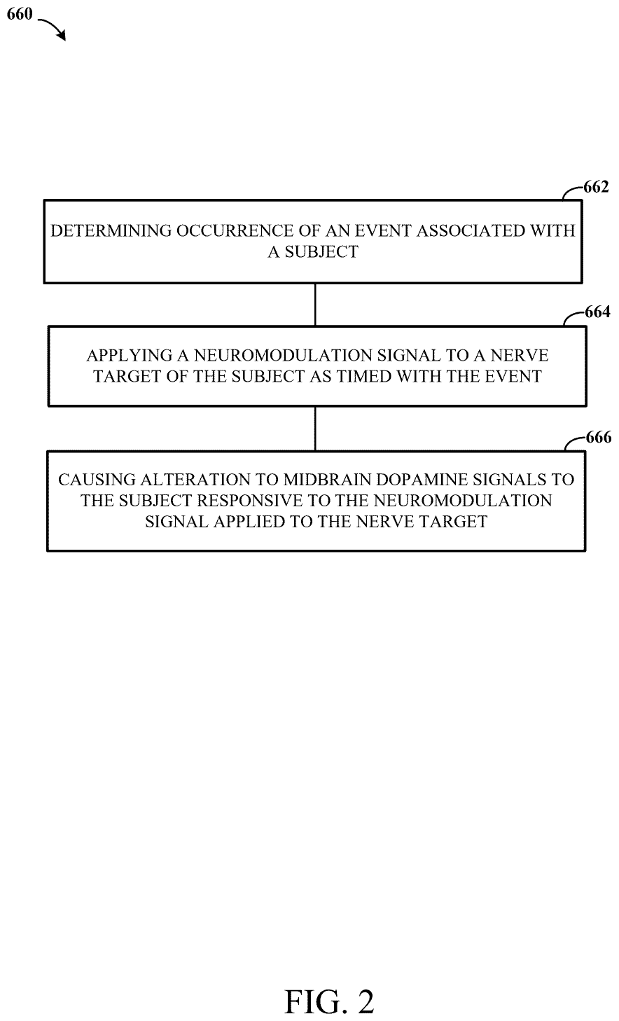

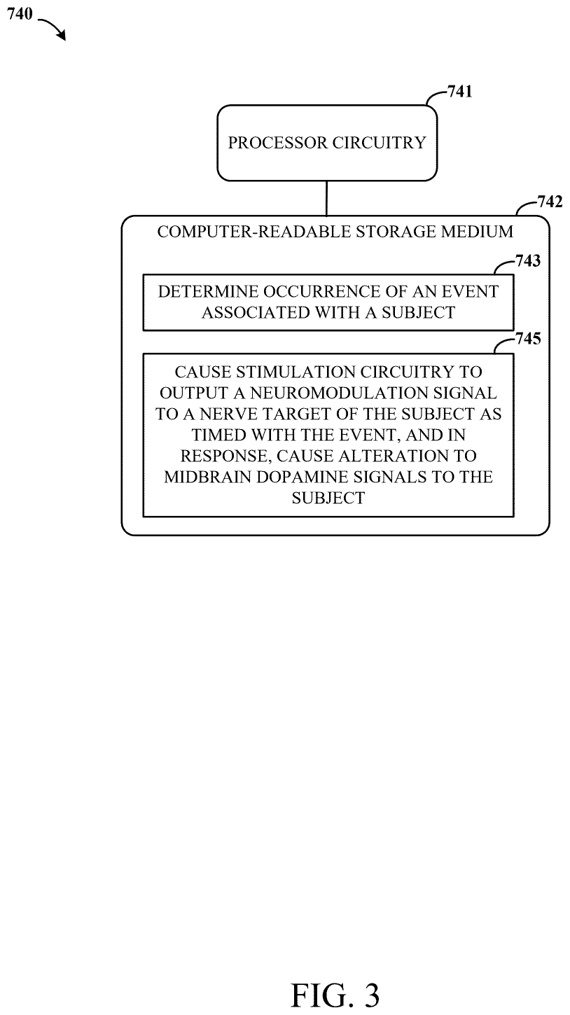

FIG. 2 illustrates an example method for altering midbrain dopamine signals, in accordance with the present disclosure. The method 660 can be implemented by the system 500 of FIG. 1 and/or the computing device 740 of FIG. 3.

At 662, the method 662 includes determining occurrence of an event associated with a subject. The occurrence of the event can be determined using a sensed signal, such as a biosignal, optical signal, acoustic signal, or other types of signals. For example, an optical signal can be received which is processed to identify an object associated with an event. As another example, a signal from an RFID tag can be received which indicates occurrence of an athletic-related event. In some embodiments, the occurrence of the event can be determined based on a user input or other input from another device indicating the event has occurred. For example, the subject may be using a computing device to learn a skill, and the computing device outputs an indication of the skill occurring. As another example, a coach can provide an input to a computing device indicating an athletic-related event has occurred, such as goal scored, which is communicated to the processor circuitry to cause stimulation circuitry to output neuromodulation signals.

At 664, the method 660 includes applying a neuromodulation signal to a nerve target of the subject as timed with the event. In some embodiments, the method 660 can include receiving data, from sensor circuitry or other communication circuitry, indicative of the occurrence of the event and, in response, determining the event has occurred and applying the neuromodulation signal within a threshold time of the event occurrence. At 666, the method 660 includes causing alteration to midbrain dopamine signals to the subject responsive to the neuromodulation signal applied to the nerve target.

In some embodiments, as previously described, the neuromodulation signal can be downloaded from external memory circuitry. For example, the method 660 can include downloading a plurality of neuromodulation signals, including the neuromodulation signal, from external memory circuitry, wherein each of the plurality of neuromodulation signals represent a processed nerve tissue signal as a sequence of at least one state corresponding to a set of state parameters and correlated with causing a particular physiological effect. In such embodiments, at least a subset of the plurality of neuromodulation signals, including the neuromodulation signal, can be correlated with activating the midbrain dopamine signal pathway to cause alteration to the midbrain dopamine signals. In some embodiments, the downloaded plurality of neuromodulation signals can be stored locally on a depository.

Various embodiment can include training and/or use of a machine learning model. In some embodiments, the method 660 includes selecting the event to time the neuromodulation signal to. In some embodiments, the event can be selected using a machine learning model which is trained to predict alteration to the midbrain dopamine signals and to predict an effect (e.g., condition improvement) in response to the neuromodulation signal applied to the nerve target. In some embodiments, an indication of the effect can be output.

In some embodiments, the method 660 includes identifying a transfer pattern that maps known neuromodulation signals to known effects using a machine learning model which is trained using an input data set including the known neuromodulation signals and the known effects. In some embodiments, the method 660 further includes using the machine learning model to select the neuromodulation signal from a depository of a plurality of neuromodulation signals based on a prediction that the neuromodulation signal is to cause alteration to the midbrain dopamine signals. As previously described, each of the plurality of neuromodulation signals can represent a processed nerve tissue signal (e.g., a neurogram) as a sequence of at least one state corresponding to a set of state parameters and correlated with causing a particular physiological effect. In some embodiments, the method 660 can further include outputting, using on the machine learning model, at least one of: (i) a predicted effect of the neuromodulation signal or an additional neuromodulation signal; (ii) the event or an additional event to time the neuromodulation signal or an additional neuromodulation signal to; and (iii) a stimulus program including an additional plurality of neuromodulation signals and events to achieve an effect.

The method 660 can include further variations and additional features, such as those described above in connection with the system 500 of FIG. 1. For example, the method 660 can include any of the features executed by the processor circuitry 502, the sensor circuitry 504, and/or the stimulation circuitry 506. The common features and attributes are not repeated for each of reference.

FIG. 3 illustrates an example computing device including non-transitory computer-readable storage medium storing executable code, in accordance with the present disclosure. In some embodiments, the processor circuitry 741 can form part of a computing device 740. The computing device 740 can include processor circuitry 741 and computer-readable storage medium 742 storing a set of instructions 743, 745. Although embodiments are not so limited, and the processor circuitry 741 and computer-readable storage medium 742 can form a part of distributed computing devices, with the distributed computing devices being in communication with each other. The computer-readable storage medium 742 can, for example, include read-only memory (ROM), random-access memory (RAM), electrically erasable programmable read-only memory (EEPROM), Flash memory, a solid state drive, and/or discrete data register sets. As used herein, “executable instructions” can be interchanged with “instructions executed by the processor circuitry”. In some embodiments, any of the computing device 740, the processor circuitry 741, and computer-readable storage medium 742 can respectively include an implementation of and/or at least some of substantially the same features and attributes as the computing device 508, the processor circuitry 502, and the memory circuitry 510 of FIG. 1.

At 743, the processor circuitry 741 can execute instructions to determine occurrence of an event associated with a subject. At 745, the processor circuitry 741 can execute instructions to cause stimulation circuitry to output a neuromodulation signal to a nerve target of the subject as timed with the event, and in response, cause alteration to midbrain dopamine signals to the subject.

Embodiments are not limited to the instructions illustrated by FIG. 3. In some embodiments, additional or alternative instructions can be stored and executed. For example, the instructions can include further variations and additional features, such as those described above in connection with the system 500 of FIG. 1. For example, the processor circuitry 741 can execute further instructions to implement any of the features executed by the processor circuitry 502 of FIG. 1. The common features and attributes are not repeated for ease of reference.

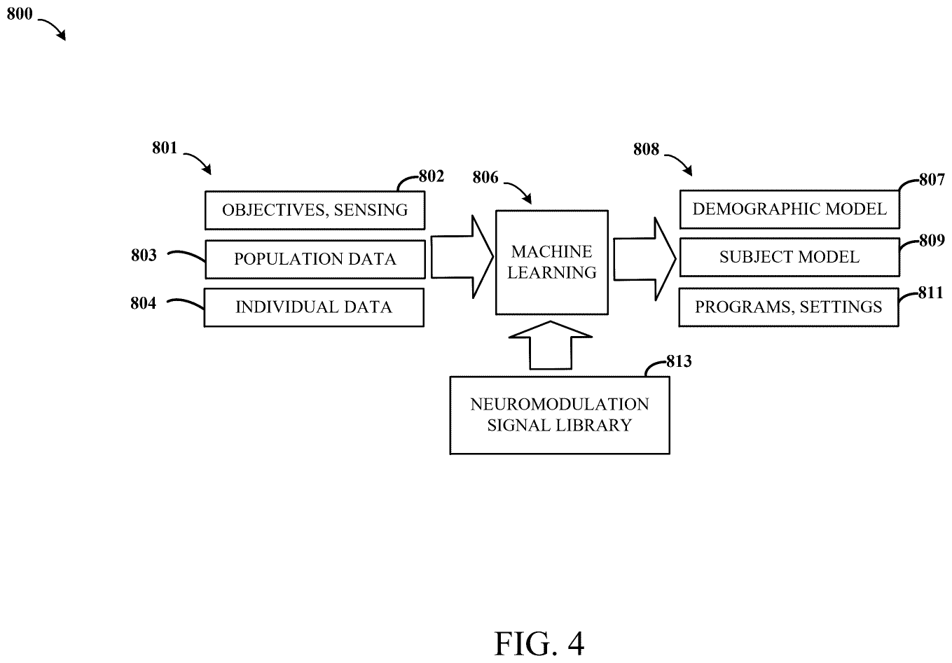

FIG. 4 illustrates an example machine learning process, in accordance with the present disclosure. More particularly, FIG. 4 shows a schematic view of an example machine learning process 800, which can be used to train different machine learning models 808. For example, different types of data sets 801 can be input for machine learning which include known inputs and known outputs for training different machine learning models 808. The data sets 801 can include objectives and sensing 802, demographic data 803 including sensed signals and neuromodulation signals (and optionally, other types of biostimulation signals), individual data 804 including sensed signals and neuromodulation signals (and optionally, other types of biostimulation signals). As previously described, the sensed signals can include biosignals which are derived from the biological state and signals derived from the environmental data. The objective can be specific effect or set of effects (e.g., goals) for the subject. The data set(s) 801 are input to machine learning layer 806 for learning from the recorded and marked data of demographic subjects for generating the demographic-machine learning model 807 and then fine-tuned to generate a subject-specific machine learning model 809. Based on objectives input (e.g., 802), the model(s) 807 and/or 809 can be updated to generate a stimulus program 811. For example, the stimulus program 811 can contain recorded neurograms (and/or other tissue signals), synthesized neuromodulation signals (and/or other biostimulation signals), scheduled timings with events, and stimulation parameter values. As previously described, a set of different neuromodulation signals (and optionally, other types of biostimulation signals) can be stored as a library 813, and subset associated with a particular effect(s) can be downloaded from the library 813 and stored locally on a depository. Different example machine learning models are further illustrated and described in connection with FIGS. 6A-6G.

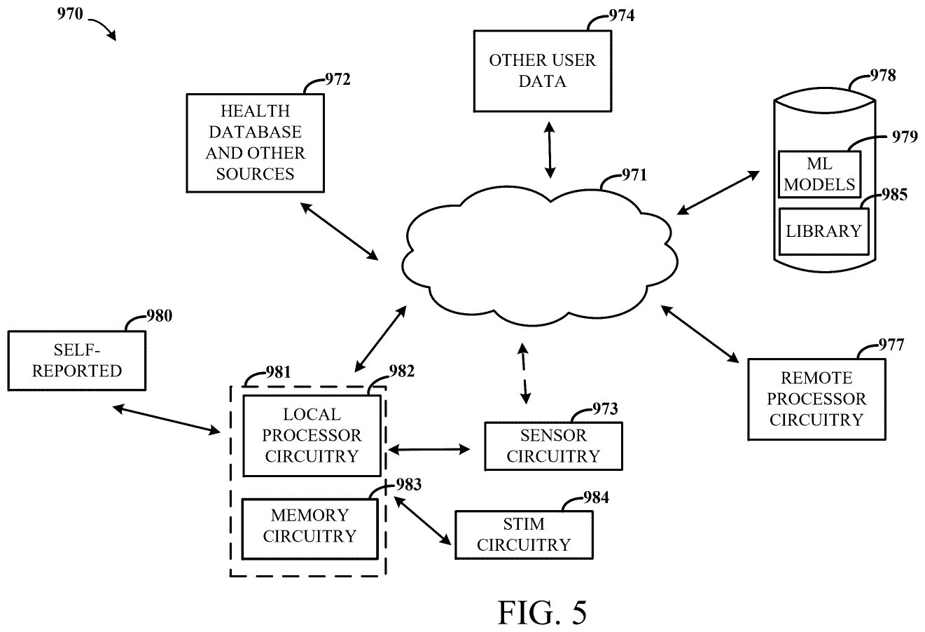

FIG. 5 illustrates an example system including machine learning models, in accordance with the present disclosure. As shown, a variety of computing resources 973, 974, 977, 978, 982, 983, 984 can form the system 970. In some embodiments, the system 970 can include a cloud computing system or other type of network system which includes a plurality of distributed computing resources. The distributed computing resources can be at different locations, such as local computing resources 982, 983, 973 which are located proximal to the subject and remotely-located computing resources 977, 978 which are located remotely from the subject. The various computing resources 973, 974, 977, 978, 982, 983, 984 can communicate with one another using data communications over a network 971. For example, the system 970 can be cloud-based and/or can be implemented through other computer systems in alternative architectures, such as a peer-to-peer network.

The system 970 includes a local processor circuitry 982 and local memory circuitry 983, and which can form part of a computing device 981. The computing device 981 can be local to the subject, such as a smartphone, laptop, desk computer, or other device that is accessible to the subject. The computing device 981 can be in communication with stimulation circuitry 984 and/or sensor circuitry 973 that is local to the subject. The memory circuitry 983 can store instructions executable by the processor circuitry 982. In some embodiments, the memory circuitry 983 can store a local version of a machine learning model or models 979 and/or portions of a library 985 which can be obtained from a back-end database 978. In some embodiments, the machine learning model or models 979 and/or portions of a library 985 can be stored temporarily on the local processor circuitry 982 (e.g., in cache memory).

In some embodiments, the machine learning model 979 is stored on the back-end database 978 and processed remotely by a remotely-located processor circuitry 977. Although one database 978 is illustrated, the system 970 can include a plurality of databases stored on memory circuits which are accessible by a plurality of distributed processor circuits which can train the machine learning models 979. For example, the remotely-located processor circuitry 977 can construct and train the machine learning model 979 and provide the trained machine learning model 979 to the local processor circuitry 982. In some such embodiments, either the local processor circuitry 982 or the remotely-located processor circuitry 977 can revise (e.g., retrain) the machine learning model 979 and/or neuromodulation signals (and optionally, other types of biostimulation signals) using tracked sensor data from the sensor circuitry 973 or other feedback data, as previously described. In other embodiments, the remotely-located processor circuitry 977 can remotely apply the trained machine learning model 979 to input data, which is communicated to the remotely-located processor circuitry 977 directly from the sensor circuitry 973 or from the local processor circuitry 982.

The system 970 can include other inputs which can be used to generate the machine learning models 979 and/or revise the trained machine learning models 979 to be subject-specific. In some embodiments, the additional inputs include self-reported measures 980, such as subject provided inputs on a condition. The self-reported measure 980 can be communicated via input circuitry to the local computing device 981. In some embodiments, the system 970 can include health databases or other sources of health information 972, such as a health application or patient portal to a professional and which allows for feedback information or self-reported measures to be input as a features to the machine learning models 979.

FIGS. 6A-6G illustrate example machine learning models, in accordance with the present disclosure. The various machine learning models can be generated and/or trained locally by local processor circuitry and/or remotely and stored on a database, such as illustrated by FIG. 5.

FIG. 6A illustrates a schematic of an example machine learning model for stimulation. In various embodiments, a machine learning model 1040 can be trained to learn a transfer pattern that maps neuromodulation signals 1041 applied and the effect 1043, as shown by 1042. As previously described, a plurality of neuromodulation signals with different values for a stimulation parameter(s) can have different effects. For example, the stimulus frequency and amplitude for VNS can result in the excitation of different groups of nerve fibers, depending on their electrical properties. VNS can modulate the midbrain dopamine pathway (and/or other pathways) to alter the dopamine signal for reinforcement learning, to amplify goal-directed behavior, and/or disrupt cue-dependent dopamine signaling. The transfer pattern can be used as a model of a nerve used to predict the effects of stimulation. In particular, the machine learning model 1040 can associate stimulation patterns by training on a combination of collected data and in silico stimulation. The machine learning model 1040 can be trained to map stimulation patterns to effects or conversely, map effects to stimulation patterns.

FIG. 6B illustrates an example machine learning model for sensing. In various embodiments and as previously described, a machine learning model can be trained to learn a transfer pattern that maps observable variables with latent variables which are difficult to observe in normal usage. For example, observable variables can include data that is readily accessible, such as a biosignal sensed 1052 through wearable devices (e.g., chest strap or smartwatch). An example observable biosignal includes ECG. In contrast, MRI data is difficult to obtain, but provides a map of brain activity during stimulation. This is an example of a latent variable. Embodiments are not limited to biosignals, and variables can include extrinsic observables, such as skill-learning visual cues.

As a specific example, machine learning can be used to learn the transfer pattern between ECG data and fMRI-derived brain activity that allows brain activity to be inferred during normal usage. This allows the cardiac signal to be used as a proxy signal for brain activity. The machine learning model 1050 is trained and validated using data from controlled and fixtured experiments that, while difficult to perform, need only be executed during a setup phase for training. This application of machine learning can be used to associate any easily observed set of observables with latent variables. For example, once trained, sensor circuitry 1054 can be used to sense ECG data (e.g., 1052) responsive to neuromodulation signals output 1053 and use the ECG data as a proxy for brain activity as the data collected 1051.

FIG. 6C illustrates an example implementation of a machine learning process to enable control of a cardiac signal through breathing. As shown, a machine learning process can involve a system 1060 including a computing device 1063 which includes or has access to a depository 1061 of neuromodulation signals and outputs neuromodulation signals, in a digital form, from the depository 1061 to stimulation circuitry, as shown at 1062. The stimulation circuitry converts the neuromodulation signal to analog form and outputs the neuromodulation signal to a target of the subject, at 1059. Sensor circuitry 1054 obtains measures of a biosignal in response to the neuromodulation signal, such as the illustrated ECG, and provides the biosignal measures to the computing device 1063, as illustrated at 1065. The collected data is output to a database 1066, as illustrated at 1065. The database 1066 can include identification of the neuromodulation signals applied, the biosignal measures, and time-stamped event data. Using the database 1066, transfer patterns can be learned between stimulation patterns, timing with events, and intended effects, as illustrated at 1067. The training data can be collected to learn stimulation patterns associated with different disease states or behavioral states. For example, the input data (e.g., training data) can be collected to learn associations between stimulation patterns and an objective function, such as a goal, response optimization, or other effect.

In some embodiments, the objective function can include generating a demographic-machine learning model that is standardized across many individuals. For example, the machine learning model can be trained to associate neuromodulation with responses in a subject-agnostic manner. The machine learning model can be trained using stimulation-sensing measurements across a population to learn transfer patterns that map from neuromodulation to effect and create subject-agnostic stimulus programs for optimizing an effect.

The different machine learning models can individually or together be used to predict the effects of neuromodulation and/or learn mappings of observable variable(s) to latent variables. For example, the machine learning models can learn the mappings between stimulation and responses that are individualized, learn stimulation sequences that optimize objective functions around sensed responses (e.g., VNS-induced dopamine scheduled to promote or inhibit behavior), and standardized these mappings across a demographic, among other techniques.

FIG. 6D illustrates an example machine learning system 1070 which can be used to optimize a particular behavior, such as for addiction, learning, and/or disorder treatment. Such a system 1070 can use Markov chains to model aspects of behavior, including but not limited to reward-seeking behavior. The Markov chains can have different topologies and state semantics, and prediction for how to get from a starting point to an intended effect.

The system 1070 includes a library of recordings 1071, such a neurograms. A neurogram can be an electrical recording representing of the state of a peripheral nerve. A neurogram can be processed, e.g., by a state machine, to generate synthetic neuromodulation signals of a processed neurogram. A state machine representation or representation can refer to a mathematical model or a numerical model of a stimulus (e.g., a computation model defined by sets of states, initial states and inputs/causes of transitions between states). In some embodiments, each state in the state machine representation corresponds to a set of state parameters that dictate a known spike (or ramp) amplitude and timing interval. For example, a processed neurogram can have an associated set of synthetic neuromodulation signals, such that application of the stimulus according to the set of state parameters can result in the known or expected spike (or ramp) amplitude and timing.

In some embodiments, a neurogram development environment (NDE) 1072 can be used to assemble neurostimulation signals from experimental and synthetic sources. The library of recordings 1071 can be input to the NDE 1072 to output synthetic neuromodulation signals 1080. The neurograms can include structured spikes and/or ramps that represent recruitment of nerve fibers to inform caudal (post-brainstem) targets about non-motor and non-sensory somatic state. As previously described, neurograms can be characterized in part by the evolution of firing rate and amplitude within the spike or ramp train. Different stimuli appear to give rise to distinct neurogram structures. The library of recordings 1071 can include neurograms recorded after placing a subject under a particular condition. The structures of different neurograms can be used to build parameterized state-machine models of synthetic neuromodulation signals to evoke particular, specific responses in a subject. The synthetic neuromodulation signals can be modified by changing the state parameters which were used to create the synthetic neuromodulation signal.

As previously described, each state in the state-machine model can define a set of state parameters that can be used to stochastically, or deterministically, generate neuromodulation signals (NMSs) in the form of a spike or ramp train of desired amplitude and rate. An NMS can be defined by a sequence of states that identify distribution parameters and duration for spike or ramp train generation at each state, along with state transitions that define duration and state-to-state interpolation methods. New neuromodulation signals can be generated at will without the need for surgery, recording, or sacrifice of animal subjects, such as described in U.S. Pat. No. 11,752,233. For example, neuromodulation is applied to elicit an intended effect and neurogram(s) are recorded in response. The neurograms are digitized by the NDE 1072 and processed using a state machine to generate the state-machine representations of the neurograms which include the at least one state parameter and can be used to generate the neuromodulation signals. The state machine editor 1074 can edit the state-machine representation based on signal segmentation 1075 and/or physiological targeting 1076. Physiological targeting includes and/or refers to the spatial effects, or possibilities thereof, arising from the choice of waveform. One example is focused ultrasound to selectively stimulate tissue. Another example is the use of specific frequency and power combinations to achieve a desired penetration depth for stimulation. The segmentation 1075 can include hand segmentation, where a user identifies state intervals, or states and segmentations are learned by a machine learning model. After segmentation, the state machine editor 1074 can compute state parameters and save the state machine descriptors. In some embodiments, a machine learning model can learn from prior data about different states and can include probabilities of a spike or ramp belonging with that state. The machine learning model can be a Markov model that learns hidden states or variables for state and/or effect, such as for physiological targeting.

The synthetic neuromodulation signals 1080 can be provided as a library from which a local computing device 1082 can selectively download a plurality of neuromodulation signals for a particular effect.

FIG. 6E illustrates example of state machines which can be used in a Markov model within the system 1070 of FIG. 6D. In particular, the state machine 1090 can include multiple threads 1092, 1094 of neuromodulation signals 1093-1, 1093-2, 1093-3, 1095-1, 1095-2, 1095-3, 1095-4 generated by each thread 1092, 1094. The neuromodulation signals 1093-1, 1093-2, 1093-3, 1095-1, 1095-2, 1095-3, 1095-4 are superposed to generate the full neuromodulation signal S1 1096 (with SO 1991 representing a starting state). The neuromodulation signals 1093-1, 1093-2, 1093-3, 1095-1, 1095-2, 1095-3, 1095-4 can be scheduled over time and in response to other events.

The state machines can be used to search for variables that optimize the neuromodulation signals. For example, different state parameters can be searched, including the number of states, statistics for the states (e.g., mean and variances for amplitude and timing), repetitions of the neuromodulation signal, duty cycle (e.g., resting time between state machine), wait time between the repetition, and the measurement of the effect. The state machines can be used as a starting point to model behavior and learning and to perturb the models to predict maximum effect.

FIGS. 6F-6G illustrate example modified autoencoders. As previously described, the modified autoencoders 1100, 1110 can encode measures of the biosignal as pre-images based on respective ones of the measures of the biosignal obtained without application of biostimulation signals, and identify a transfer pattern that maps the plurality of biostimulation signals and the measures of the biosignal using the pre-images and the plurality of biostimulation signals.

As noted above, embodiments are not limited to neuromodulation signals, and may be applied to other types of biostimulation signals. In some embodiments, combinations of neuromodulation signals and other types of biostimulation can be used. Similarly, targets are not limited to nerves. For example, a target can include a nerve, differentiated tissue, cells, a muscle, and/or an organ, among other targets. As some specific examples, the biostimulation can include stimulating muscles, such as via stimulating efferent nerve fibers (e.g., Transcutaneous electrical nerve stimulation (TENS) or direct muscles stimulation (e.g., electrical muscle stimulators (EMS)). As another example, ultrasound signals can be applied to internal organs, such as to the spleen or liver. As a further example, transcranial magnetic stimulation can be applied to the brain and/or brainstem. Other biostimulation signals and/or targets can be used.

Applications for biostimulation can use biostimulation signals to achieve an effect as measurement by a variety of devices. Typical effects of the biostimulation signal are modified by the tissue and individual variation. The modified autoencoders 1100, 1110 can adapt to these variation and learn the transfer function that maps the biostimulation signals to the measured physiological effect. The modified autoencoders 1100, 1110 are used to learn the pre-images (e.g., internal representations) for the biosignal measurements in a first training (e.g., calibration period). Following the first training, a second training (e.g., second calibration period) is used to: (i) predict the effect of additional biostimulation signals on the biosignal and (ii) predict additional biostimulation signals that cause a target biosignal measurement.

The modified autoencoders 1100, 1110 can include computer-readable instructions which are stored on memory circuitry and/or executed by processor circuitry, such as the processor circuitries 502, 741 illustrated by any of FIGS. 1 and 3.

The modified autoencoders 1100, 1110 can learn from a few historical data points what are the normal signals using a large set of features (e.g., hundred(s), thousand(s)) as input data. The input data can include biosignal measures and/or biostimulation signals. The modified autoencoders 1100, 1110 can be trained to learn and memorizes the typical patterns, and includes an encoder/decoder pair with an additional encoder or decoder. For example, the encoder can take an input waveform (e.g., biosignal measures or biostimulation signals) and compress the input waveform to a compressed version, referred to as pre-images or the “latent space”. The decoder takes the pre-images and reconstructs the input waveform.

FIG. 6F illustrates an example modified autoencoder 1100 which includes a first encoder 1102, a decoder 1104, and a second encoder 1106. The modified autoencoder 1100 is first trained to learn the pre-images for a biosignal measurement. For example, the biosignal measurement is input as a waveform 1101 to the first encoder 1102 which compresses the waveform 1101 to the pre-image 1103 which is a low dimensional hidden state having reduced features as compared to the input waveform 1101. The features available can be jointly represented as information with a low dimensional hidden state that includes subsets of the features which are combined into pseudo-features of a reduced number from the input feature set, which can be referred to as an autoencoder bottleneck. The input features are compressed into the reduced number of pseudo-features by applying a combinatorial function to the subsets of the features, with the combinatorial functions being learned during the first training of the modified autoencoder 1100 by comparing the input features to the reconstructed features and adjusting the combinatorial functions to reduce a reconstruction error. As such, the bottleneck layer can include fewer pseudo-features than the input features, which are output to the decoder 1104. The first encoder 1102 thereby reduces the size of the input waveform 1101 and reduces the size of the input task by combining subsets of the input features into pseudo-features that represent the respective subsets of the input features.

The decoder 1104 uses the hidden information to reconstruct the original input waveform 1101 from the pre-image 1103, as shown be the reconstructed waveform 1107. The hidden information can include the pseudo-features and the combinatorial functions used to compress or generate the pseudo-features.

After the first training, the second encoder 1106 is trained to learn a transfer pattern that maps the stimulus space to the pre-image 1103 of the biosignal measure. For example, the transfer pattern can map an input biostimulation signal waveform 1105 to the pre-image 1103 of the biosignal measure. In some embodiments, a loss transfer pattern 1109 can be learned which can be used to predict biosignal properties from the biostimulation signal.

FIG. 6G illustrates another example modified autoencoder 1110 which includes an encoder 1102, a first decoder 1104, and a second decoder 1112. The modified autoencoder 1110 is first trained to learn the pre-images 1103 for a biosignal measurement (e.g., 1101) using the encoder 1102 and first decoder 1104, as previously described in connection with FIG. 6F.

After the first training, the second decoder 1112 is trained to learn a transfer pattern that maps the pre-image 1103 of the biosignal measure into the stimulus space. For example, the transfer pattern can map the pre-image 1103 of the biosignal measure to a predicted biostimulation signal 1111. A loss transfer pattern 1113 can be learned which can be used to predict a biostimulation signal that can produce a biosignal measurement.

In some embodiments, the modified autoencoders 1100, 1110 can include a modified variational autoencoder (VAE) that identifies a similarity of a plurality of tasks (e.g., the pseudo-features) to an input task (e.g., analyzing the set of features). For example, a VAE can include at least one encoder, such as set of encoders which reduce a size of the input tasks to a smaller latent space z, and at least one decoder, such as a set of decoders which reconstructs the latent space z (e.g., a cluster of related tasks which are reduced to the latent space) into an output task representative of the cluster of related tasks.

In some embodiments, the modified autoencoders 1100, 1110 can depend on at least the features to be processed and in some embodiments can include at least one of a vanilla autoencoder, a VAE, and a VAE for time series anomaly detection. In some embodiments, an encoder 1102 of the modified autoencoders 1100, 1110 can implement a neural network, such as convolution neural network a long short-term memory (LSTM), to convert the received input set of features into the compressed form, e.g., pre-images.

In some embodiments, the pre-images 1103 are parameterized. For example, the pre-images 1103 can use latent space parameterized derived from stimulation parameters. The machine learning model can be trained to coerce the pre-images 1103 to have a structure that is homeomorphic to the stimulus parameterization. In some embodiments, instead of using discrete stimulation/no-stimulation or state labels, labels are supplied that are continuous and are derived from smooth mappings from stimulation parameter space into the autoencoder latent space. Assumption is that normally-distributed noise “follows” the point in parameter space as stimulus progresses.

Various embodiments are directed to expanding the biostimulation signal space by sweeping stimulation parameters to or through a plurality of different values, as noted in various embodiments above. For example, the system 500 of FIG. 1 can alternatively or additionally be used to expand the biostimulation signal space. Biostimulation signals, including neuromodulation signals, typically take form of a square wave pulse train with specific frequencies, duty cycles, and current. In some cases, there is an on-off pattern that takes form in an on period for fixed number of cycles and an off period (e.g., similar to wash periods) for a different interval, and with a certain number of repetitions. A limitation of such an approach is that it considers a small subset of an effectively infinite signal space that can contain many useful stimuli and runs the risk of side effect or off-target effect.

Embodiments in accordance with the present disclosure use a fuzzing technique of computer science as applied to the problem of stimuli for different intended purposes. The system assumes the sensor input (e.g., ECG) and stimuli in various forms (e.g., acoustic nerve stimulation, electrical nerve stimulation).

Embodiments assume that the signal space is generated by stimulation parameters that can control the stimulus. The stimulation parameters include pulse frequency, amplitude, duration, progression as a function of time, (arbitrary) waveform shapes, and delivery portal (e.g., left-side, right-side, bilateral, ear, chest, eyes). The general parameter space is chosen to search for the space of possible signals and then this parameter space is sampled. Each sample corresponds to stimulation that is applied to a subject. The biosignal response of the subject is recorded and compared to biosignal response to other stimulation to find an optimal signal space that achieves the intended effects. The approach can be generalized to multiple subjects and delivery portals (e.g., auricular, cervical, sacral). Further, the approach expands the signal space to the space of all piecewise continuous functions and also samples the signal space in organized way (e.g., by chirping the parameters) to optimize effects as a function of the biostimulation signal.

To the extent that subject responses vary across individuals, the system can provide a path to biometric protection of stimulation signals that are designed for individual subject response.

In some such embodiments, the stimulation circuitry is configured to output a biostimulation signal to a target of a subject and the sensor circuitry is configured to obtain measures of a biosignal from the subject in response to the biostimulation signal output to the target. The processor circuitry can be configured to: (i) select at least two stimulation parameters and a plurality of values for the at least two stimulation parameters; (ii) cause the stimulation circuitry to output the biostimulation signal to the target which sweeps each of the at least two stimulation parameters to the plurality of different values to sample a biostimulation signal space; and (iii) output stimulation parameter ranges for the at least two stimulation parameters that optimize a physiological effect as a function of the biostimulation based on the measures of the biosignal received from the sensor circuitry. Embodiments are not so limited, and in some instances, a single parameter may be swept to a plurality of values. In such embodiments, the biostimulation signal output to the target can include different values for the stimulation parameter.

As previously described, the plurality of stimulation parameters can be selected from: pulse frequency, duration, amplitude, duty cycle, pulse width, progression as a function of time, signal shape, and a delivery portal. And the biostimulation signal can include an electrical stimulation signal, an acoustic stimulation signal, a magnetic stimulation signal, or a combination thereof. In some embodiments, the intended physiological effect includes a change in ECG, beat frequency, HRV, HR, or gastric motility, as well as combinations thereof and various other effects.

As used herein, a neurogram includes and/or refers to a measurement of the signals that traverse a nerve. In one embodiment, the neurogram may be produced in response to application of a particular condition of a subject. It should be realized that use of the term “subject”, as used herein, includes any animal or human subject that is put under a condition and provides a neurogram and/or other biosignals. For example, the condition may be wherein the subject has been given a particular treatment, such as by administration of a drug. One example of a neurogram includes a structured sequence of electrical neuronal spikes (or ramps), where the sequence of electrical neuronal spikes (or ramps) has a characteristic amplitude envelope, an inter-spike (or ramp) interval profile and a definite extent in time (e.g., a defined time interval). A neurogram can be an electrical recording representing of the state of a peripheral nerve. A neurogram can be processed by, e.g., a finite state machine to generate synthetic neuromodulation signals of a processed neurogram.

As used herein, state machine representation or finite state machine representation may refer to, e.g., a mathematical model or a numerical model of a stimulus. In some embodiments, each state in the state machine representation corresponds to a set of state parameters that dictate a known spike or ramp amplitude and timing interval. For example, a processed neurogram can have an associated set of synthetic neuromodulation signals, such that application of the stimulus according to the set of parameters can result in the known or expected spike or ramp amplitude and timing.

The skilled artisan would recognize that various terminology as used in the Specification (including claims) connote a plain meaning in the art unless otherwise indicated. As examples, the Specification describes and/or illustrates aspects useful for implementing the claimed disclosure by way of various circuits or circuitry which may be illustrated as or using terms such as blocks, modules, device, system, unit, controller, and/or other circuit-type depictions. Such circuits or circuitry are used together with other elements to exemplify how certain embodiments may be carried out in the form or structures, steps, functions, operations, activities, etc. For example, in certain of the above-discussed embodiments, one or more modules are discrete logic circuits or programmable logic circuits configured and arranged for implementing these operations/activities, as may be carried out in the approaches shown in FIG. 1. In certain embodiments, such a programmable circuit is one or more computer circuits, including memory circuitry for storing and accessing a program to be executed as a set (or sets) of instructions (and/or to be used as configuration data to define how the programmable circuit is to perform), and an algorithm or process as described at FIG. 3 is used by the programmable circuit to perform the related steps, functions, operations, activities, etc. Depending on the application, the instructions (and/or configuration data) can be configured for implementation in logic circuitry, with the instructions (whether characterized in the form of object code, firmware or software) stored in and accessible from a memory (circuit).

Various embodiments are implemented in accordance with the underlying Provisional applications: (i) Provisional Application No. 63/646,062, filed on May 13, 2024, and entitled “Systems and Methods for Discovery of Bioelectric Stimuli”; and (ii) Provisional Application No. 63/646,066, filed on May 13, 2024, and entitled “Systems for Alleviating Addiction Through Vagus Nerve Stimulation”, to which benefit is claimed and each of which is fully incorporated herein by reference in its entirety for its general and specific teachings. For instance, embodiments herein and/or in the Provisional Applications can be combined in varying degrees (including wholly). Reference can also be made to the experimental teachings and underlying references provided in the underlying Provisional Applications. Embodiments discussed in the Provisional Applications are not intended, in any way, to be limiting to the overall technical disclosure, or to any part of the claimed disclosure unless specifically noted.

### EXPERIMENTAL EMBODIMENTS

Various experimental embodiments were directed to stimulating a nerve target as timed with an event to cause alteration to midbrain dopamine signals for causing a particular physiological and/or behavioral effect. Some experimental embodiments were directed to assessing and expanding the signal space by chirping stimulation parameters to optimize a physiological effect. In various embodiments, different machine learning models were trained and applied to input data. Some experimental embodiments were directed to applying taVNS to the left side, the right side, and/or bilaterally (e.g., left and right sides) and observing the differences.

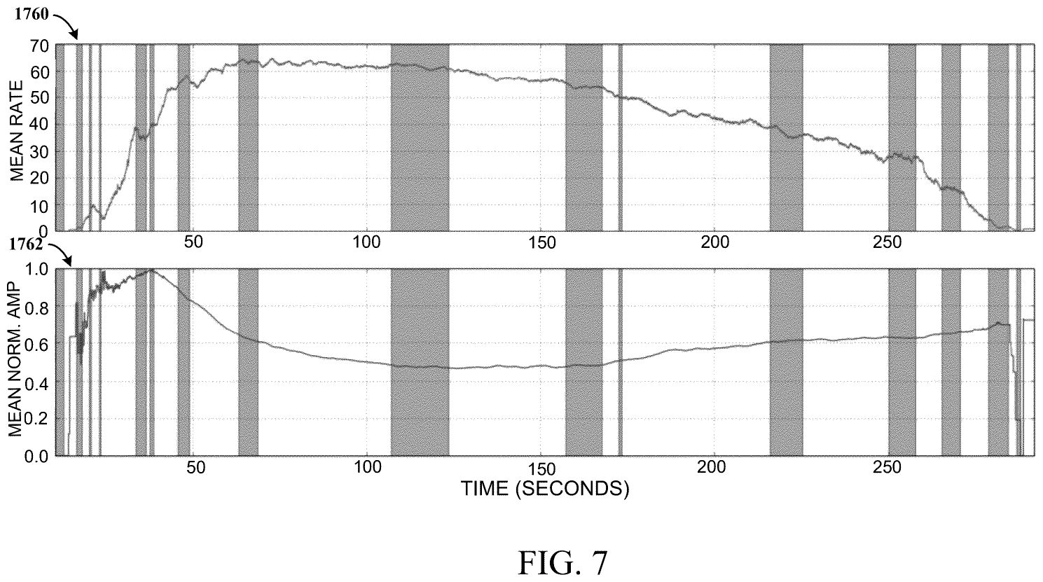

FIG. 7 illustrates example neurogram recordings, in accordance with the present disclosure. As shown, the NDE can display, on a user interface, a representation of a raw neurogram recording with states highlighted in green. The top graph 1760 includes mean firing verse time and the bottom graph 1762 includes normalized spike amplitude verses time after state machine editing. For example, a user and/or machine learning model identifies states (in green) and the NDE computes state parameters and save state machine description encoding the state parameters. As previously described, states are generative statistical models. Each state (and transition) can be used as a classifier for spikes of the neurogram. Given one neurogram, a multi-state-machine can be derived by setting k=1 and iterate: (i) user identifies state intervals and creates state machine Sₖ, (ii) NDE classifies spikes using the S and marks all spikes explained by Sₖ, (iii) remove marked spikes from consideration, and (iv) while remaining spikes, increment k and go to 1. The set {Sₖ} can contain a more thorough input neurogram. Embodiments are not so limited and the above can be applied to ramps.

Various experiments were directed to assessing stimulus-induced performance for various learned tasks to show stimulation improved learning. Some experiments were directed to directing subjects to navigate from one corner to another on a user interface through a hidden, random maze. The subjects were shown a graphic user interface of blank squares, with a visual pattern shown before each move. The pattern predicts the correct move and the subject must learn the association between the patterns and the direction for the move. The experimental results showed that electrical stimulation to the right ear, but not left ear, resulted in accelerated learning and improved performance. The performance improvement was dependent on the stimulation parameters of pulse width and frequency, duration of stimulation, and timing of stimulation with respect to the event.

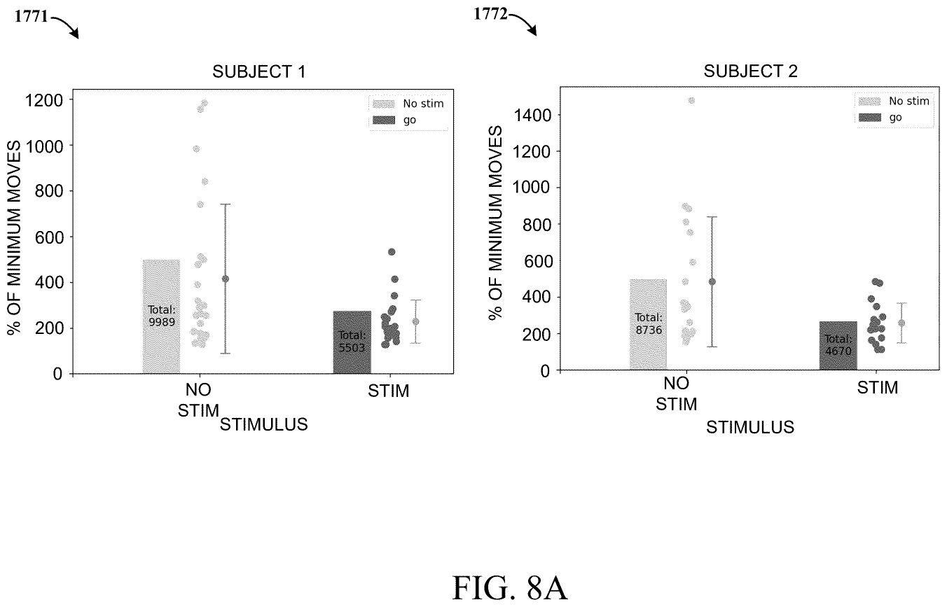

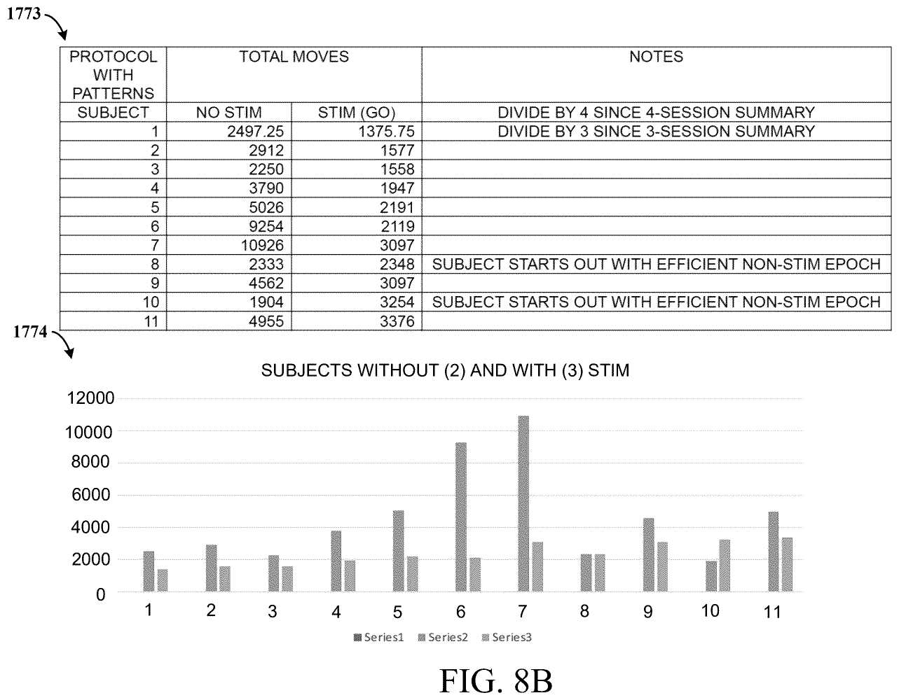

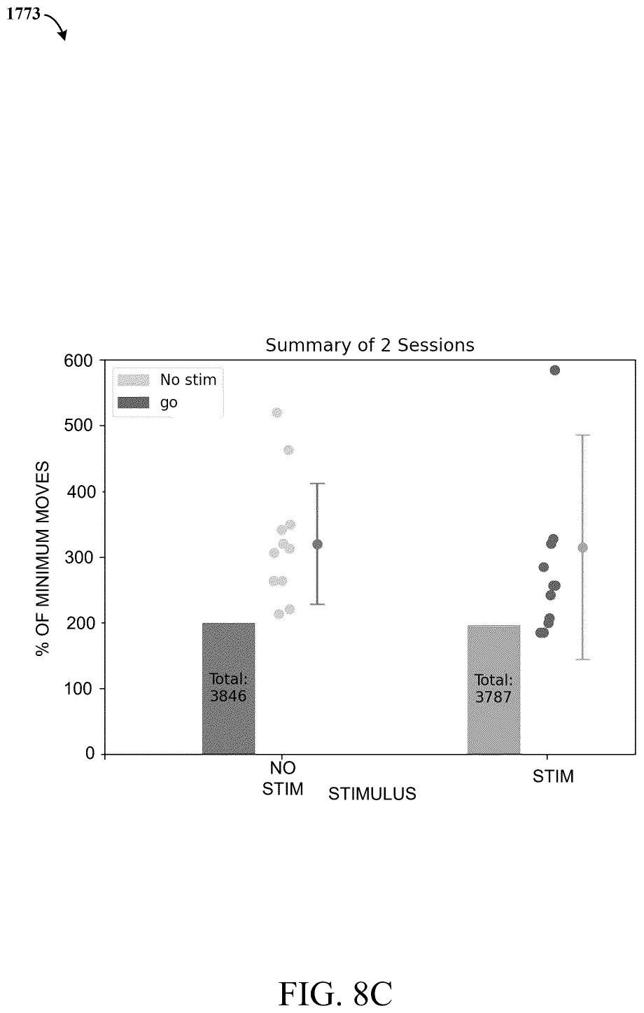

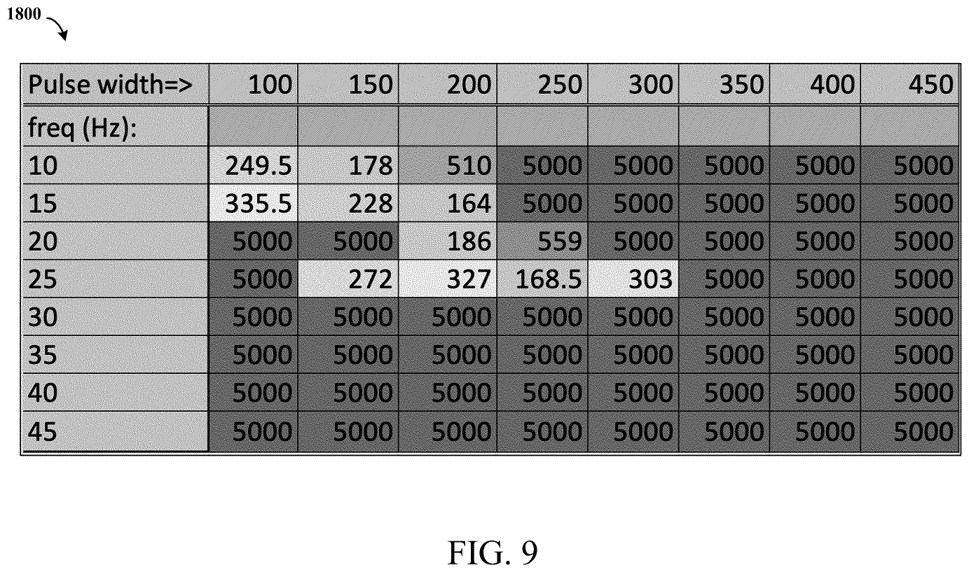

FIGS. 8A-9 illustrate example results of altering midbrain dopamine signals, in accordance with the present disclosure. The stimulation was applied while subjects were playing the hidden, random maze, as described above. FIG. 8A illustrates graphs 1771, 1772 showing the results of two example subjects with stimulation and without stimulation, showing a decrease in moves without stimulation. The stimulation included a 150 ms biphasic “gaussian” pulse delivered at 15 Hz. FIG. 8B shows the results of many subjects (table 1773 and chart 1774). The chart 1774 shows non-stimulation results in orange and stimulation in grey. Subject 8 and 9 did worse with stimulation, which may be due to the subjects doing well without stimulation. VNS tended to speed up learning, with consistency being reached within 10 trials, sometimes within 2 trials. Without VNS, performance converged more slowly and was not consistent. The neuromodulation applied to obtain the data in FIGS. 8A-8B was right-side taVNS. FIG. 8C shows the result from a subject subjected to left-side taVNA. As shown in FIG. 8C, the results of stimulation via taVNA and no-stimulation were similar and had a close mean percentage.

FIG. 9 is a heat map showing stimulation parameter sensitivity. A higher number indicated the player made more moves than the minimum. The numbers within the map 1800 represent the percentage of minimum path with 100 (e.g., 100 percent) being the most efficient and representing a subject making no unnecessary moves.

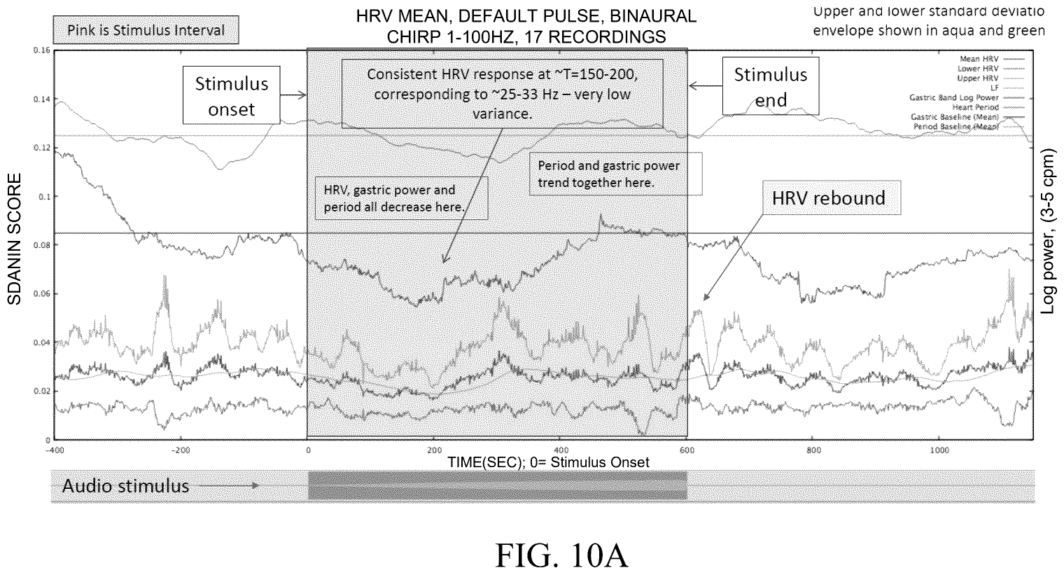

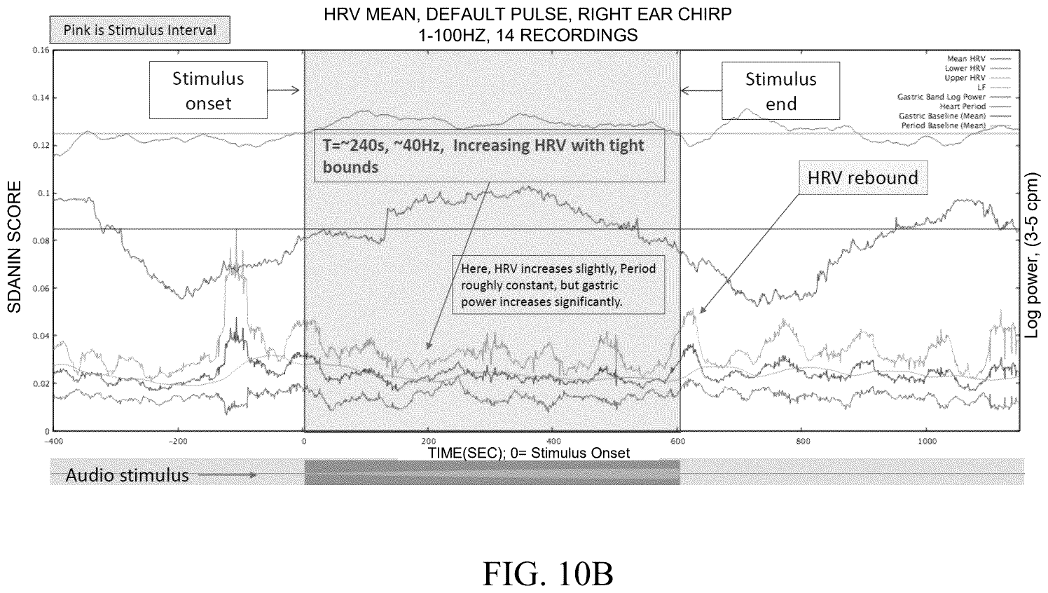

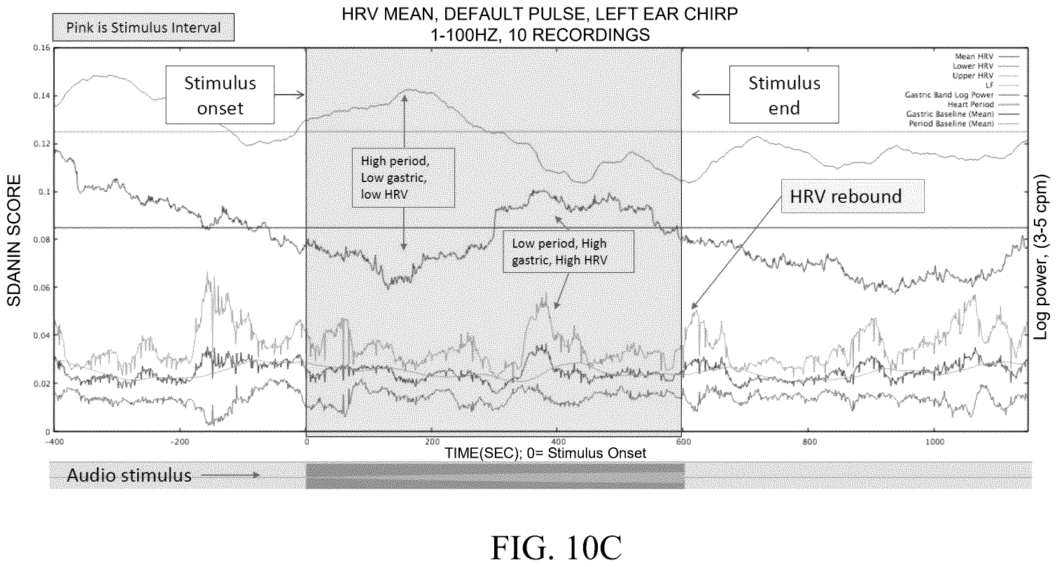

Various experiments were directed to expanding the signal space by sweeping a stimulation parameter over a plurality of values and observing the response. In a specific embodiment, the biostimulation signal included a ten minute signal in which the signal is swept from 1-100 Hz. In response, HRV was measured. FIGS. 10A-10C illustrate example HRV measures responsive to the biostimulation signal (in pink) as applied bilaterally to the vagus nerve (FIG. 10A), as applied to the right-side vagus nerve (FIG. 10B), and as applied to the left-side vagus nerve (FIG. 10C). As shown, the bilateral, left-side, and right-side stimulation exhibited different response profiles. HRV, heart period, and gastric power are correlated, but sometimes in antisense. This supports that different stimulation parameters regimes manifest distinct physiological effect.

Other experiments were similarly used to assess for other variations, such as 25 Hs binaural and interbeat power, among other variations.

Various experiments were directed to training a modified autoencoder. Such experiments included window size of 1024 samples long, centered on a single R peak, scaled to [−1,1] and zero-mean, single dataset has around 1700 heartbeats, 4-dimensional latent space (e.g., the pre-images), conditional: train with labels, creating multiple distributions in the pre-images, use the below described, Loss Function (unconventional, but effective), suitable for spike-like (or ramp) data, such as ECG and neuronal recording.

In some experiments, the following loss function was used for ECG:

waveform_loss+KL_loss

Waveform loss assigns large weight to R peaks and weak weight to lower amplitude variations: penalizes temporal misalignment of peaks. KL loss is a standard variational loss to encourage normal distributions in the latent space.

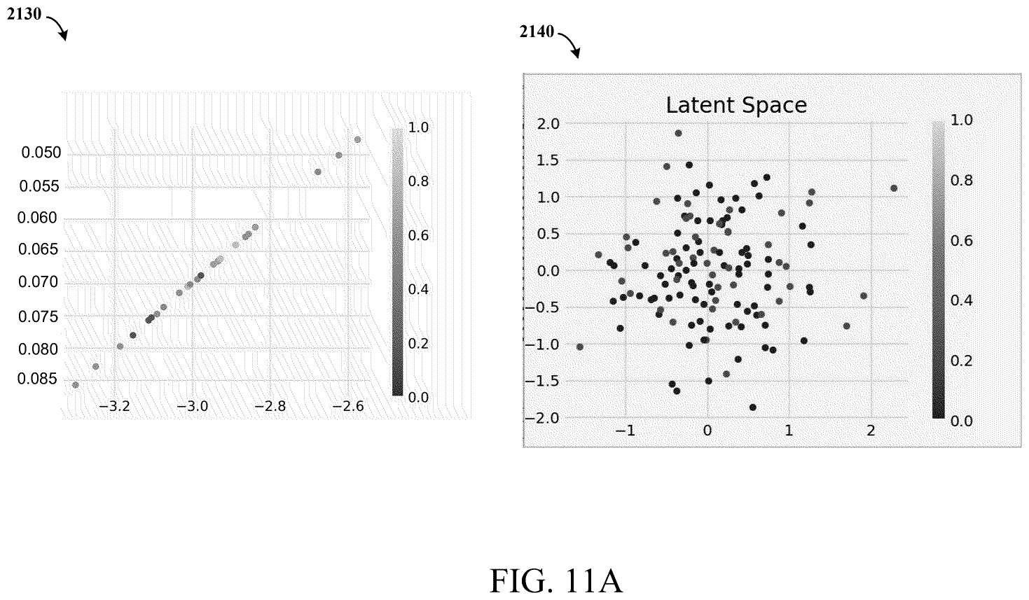

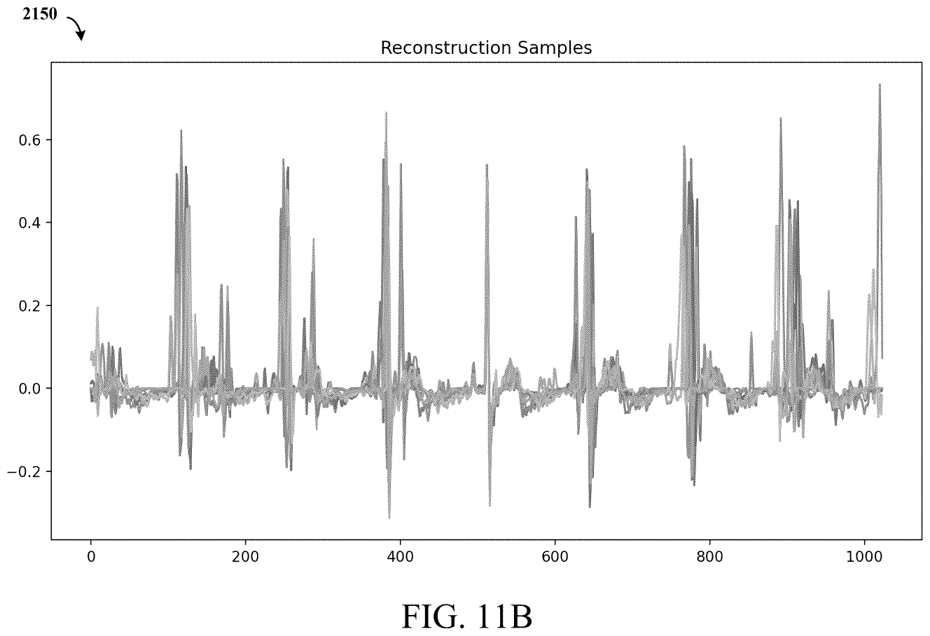

FIGS. 11A-11B illustrate example pre-images and cardiac signals output using a modified autoencoder, in accordance with the present disclosure. In FIG. 11A, a scatter plot 2130 of the latent space is a representation of a pre-image for the biosignal waveform using an AE. The scatter plot 2140 of the latent space is a representation of the pre-image for the waveform using a VAE. The VAEs forced this space to have the structure of a normal distribution. The graph 2150 of FIG. 11B shows reconstructed waveforms from a VAE trained on a single data set. The waveforms were generated by randomly picking points in the pre-images and decoding each point to a waveform.

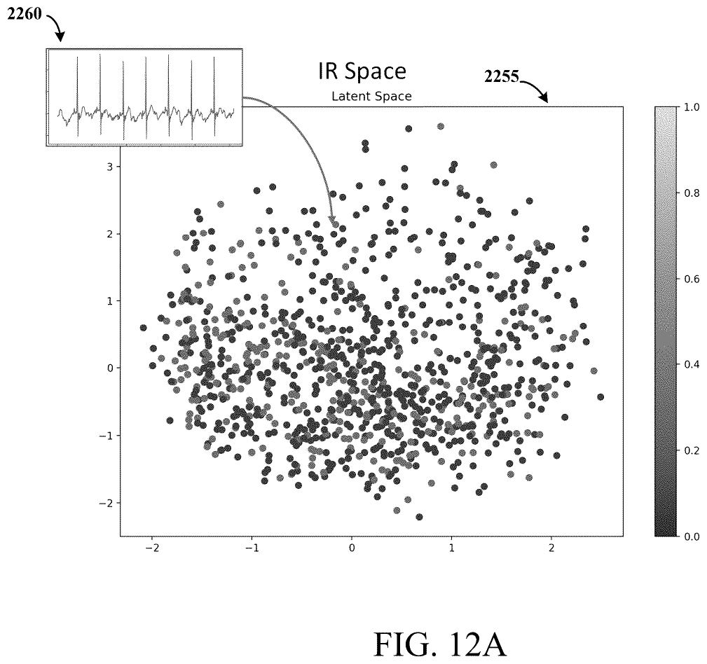

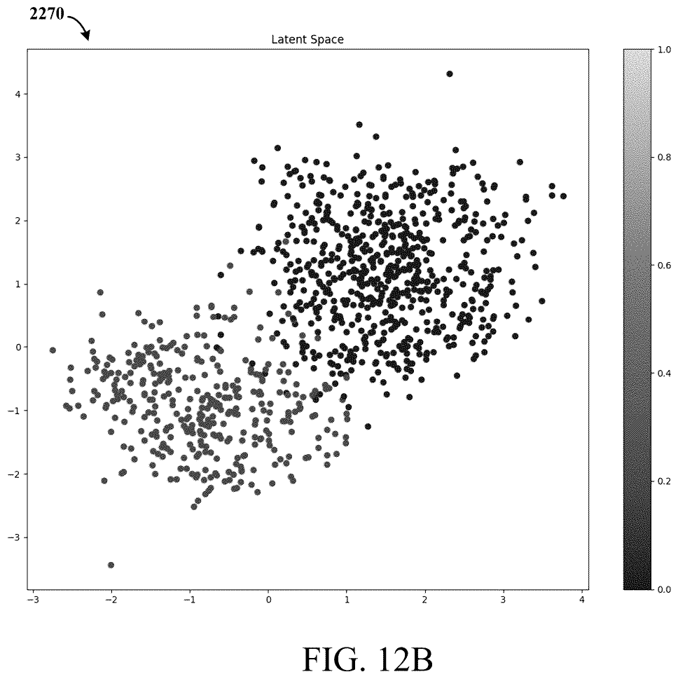

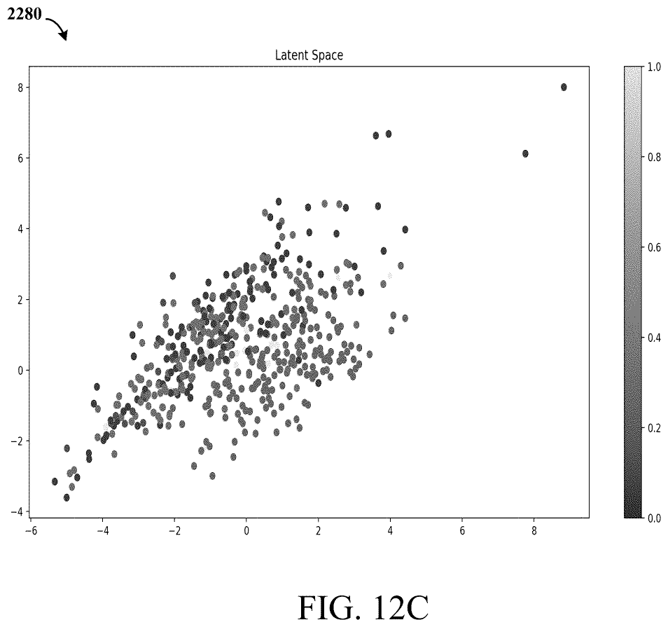

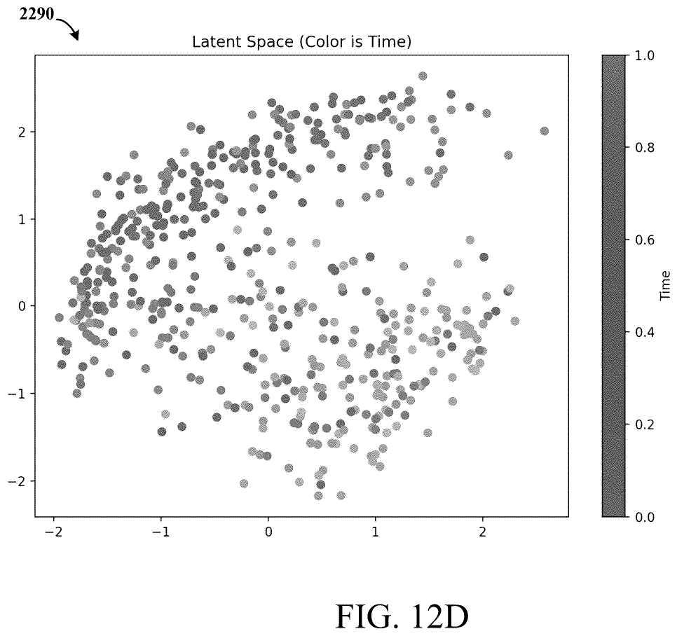

FIGS. 12A-12D illustrate example pre-image representations, in accordance with the present disclosure. FIG. 12A is a scatter plot 2255 of two out the four dimensions in the latent space. Points are generated by selecting at random about 100 real cardiac waveform windows from the dataset and encoding each one. Red dots correspond to stimulus-on ECG waveforms, as shown by 2260, and blue dots correspond to stimulus-off ECG waveforms. There can be separation between these classes. A conditional VAE: forces different labels into different distributions in latent space. FIG. 12B is a scatter plot 2270 from a conditional VAE that forces separate distributions for stimulation (red) and non-stimulation (blue) waveforms. Using the training label, the means was biased in the latent space so that the distributions are separated. In some embodiments, one-hot encoding can be used to classify waveforms conditional on stimulation. FIGS. 12A and 12B were obtained using training data.

FIG. 12C is a scatter plot 2280 from a condition VAE using non-training data. The data includes pre-stimulation (green), during stimulation (red), post-stimulation (blue), and transitional (yellow). FIG. 12C is a proof of concept that the VAE can classify new data. The stimulation and post-stimulation appeared to fall into the same distribution, which suggests that effects of stimulation linger even after stimulation is off.

Various embodiments were directed to validating a conditional VAE that was trained on stimulation and ECG data. FIG. 12D is a scatter plot 2290 on biosignal waveforms drawn at random from three recordings not used for training. Each waveform is encoded into the four-dimensional latent space. Two dimensions were plotted in the particular example. All datasets have a thirty minute duration. Stimulus generally appears at ten minutes (t=˜0.33). Color indicates relative time within the dataset: green=time 0, red=time 1 (e.g., 30 minute). Separation is a byproduct of conditional training with labels.

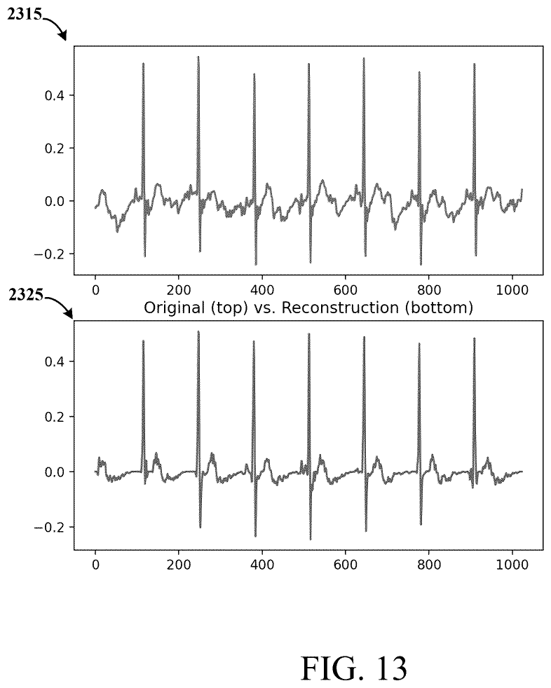

FIG. 13 illustrates an example of a measured biosignal and a reconstructed biosignal using a machine learning model (e.g., a modified autoencoder), in accordance with various embodiments. In FIG. 13, the top graph 2315 is the measured biosignal and the bottom graph 2325 is the reconstructed biosignal. The x axis is sample number: all data are windowed at 1024 samples at a sample rate of 130 Hz with around 8 seconds per window.

Although specific embodiments have been illustrated and described herein, a variety of alternate and/or equivalent implementations can be substituted for the specific embodiments shown and described without departing from the scope of the present disclosure. This application is intended to cover any adaptations or variations of the specific embodiments discussed herein.

1. A system comprising:
stimulation circuitry configured to output a neuromodulation signal to a nerve target of a subject; and
processor circuitry configured to cause the stimulation circuitry to output the neuromodulation signal to the nerve target as timed with an event, and in response, cause alteration to midbrain dopamine signals to the subject.

2. The system of claim 1, further including memory circuitry in communication with the processor circuitry which stores a depository of a plurality of neuromodulation signals, including the neuromodulation signal, wherein each of the plurality of neuromodulation signals represent a processed nerve tissue signal as a sequence of at least one state corresponding to a set of state parameters and correlated with causing a particular physiological effect, and
wherein at least a subset of the plurality of neuromodulation signals, including the neuromodulation signal, are correlated with activating a midbrain dopamine signal pathway to cause alteration to the midbrain dopamine signals.

3. The system of claim 1, wherein the processor circuitry is configured to select the event using a machine learning model that predicts alteration to the midbrain dopamine signals and predicts a condition improvement in response to the output of the neuromodulation signal.

4. The system of claim 1, wherein the processor circuitry includes a machine learning model, which is trained using an input data set including known neuromodulation signals and known effects on the midbrain dopamine signals responsive to the known neuromodulation signals, to identify a transfer pattern that maps the known neuromodulation signals to the known effects.

5. The system of claim 4, wherein the processor circuitry is configured to apply the machine learning model to additional input data to predict a particular neuromodulation signal that is to cause alteration to the midbrain dopamine signals.

6. The system of claim 4, wherein the input data set includes at least one of:
applied neuromodulation signals and indication of alteration to the midbrain dopamine signals for the subject;
applied neuromodulation signals and indication of alteration to the midbrain dopamine signals for a plurality of other subjects;
indication of the applied neuromodulation signals for the subject or for the plurality of other subjects resulting in an intended effect; and
timing of the applied neuromodulation signals for the subject or for the plurality of other subjects, and an event.

7. The system of claim 6, wherein the indication of alteration to the midbrain dopamine signals for the subject or plurality of other subjects includes at least one of:
a biosignal used as a proxy for dopamine signaling;
brain signals indicative of midbrain dopamine spikes; and
feedback from the subject.

8. The system of claim 1, wherein the processor circuitry is configured to establish a stimulus program including a sequence of a plurality of additional neuromodulation signals as timed with different events and to cause the stimulation circuitry to output the plurality of additional neuromodulation signals as timed with and in response to the different events to achieve a goal.

9. The system of claim 1, wherein the processor circuitry is configured to cause the stimulation circuitry to output the neuromodulation signal within a threshold time of the event.

10. The system of claim 1, wherein the processor circuitry is configured to cause the stimulation circuitry to output the neuromodulation signal as timed with the event and to cause alteration to the midbrain dopamine signals to cause at least one of:
dilution of an addiction cue-related reward; and
manipulation of a consumption-related reward or other cue-related reward.

11. The system of claim 1, wherein the processor circuitry is configured to:
select at least two stimulation parameters and a plurality of values for the at least two stimulation parameters;
cause the stimulation circuitry to output an additional neuromodulation signal to the nerve target which sweeps each of the at least two stimulation parameters to the plurality of values to sample a neuromodulation signal space;
determine stimulation parameter ranges for the at least two stimulation parameters that optimize alteration to the midbrain dopamine signals for the subject as a function of the additional neuromodulation signal based on measures of a biosignal received from sensor circuitry responsive to the additional neuromodulation signal; and
cause the stimulation circuitry to output the neuromodulation signal that is characterized by the at least two stimulation parameters within the determined stimulation parameter ranges.

12. The system of claim 1, wherein the processor circuitry includes a machine learning model trained to:
encode a plurality of measures of a biosignal as pre-images based on respective ones of the plurality of measures of the biosignal obtained without application of neuromodulation signals, wherein the biosignal is associated with the midbrain dopamine signals; and
identify a transfer pattern that maps a plurality of additional neuromodulation signals and the plurality of measures of the biosignals using the pre-images and a plurality of additional neuromodulation signals.

13. A method comprising:
determining occurrence of an event associated with a subject;
applying a neuromodulation signal to a nerve target of the subject as timed with the event; and
causing alteration to midbrain dopamine signals to the subject responsive to the neuromodulation signal applied to the nerve target.

14. The method of claim 13, further including downloading a plurality of neuromodulation signals, including the neuromodulation signal, from external memory circuitry, wherein each of the plurality of neuromodulation signals represent a processed nerve tissue signal as a sequence of at least one state corresponding to a set of state parameters and correlated with causing a particular physiological effect, and
wherein at least a subset of the plurality of neuromodulation signals, including the neuromodulation signal, are correlated with activating a midbrain dopamine signal pathway to cause alteration to the midbrain dopamine signals.

15. The method of claim 13, further including selecting the event using a machine learning model which is trained to predict alteration to the midbrain dopamine signals and to predict a condition improvement in response to the neuromodulation signal applied to the nerve target.

16. The method of claim 13, further including identifying a transfer pattern that maps known neuromodulation signals to known effects using a machine learning model which is trained using an input data set including the known neuromodulation signals and the known effects.

17. The method of claim 16, further including using the machine learning model to select the neuromodulation signal from a depository of a plurality of neuromodulation signals based on a prediction that the neuromodulation signal is to cause alteration to the midbrain dopamine signals, wherein each of the plurality of neuromodulation signals represent a processed nerve tissue signal as a sequence of at least one state corresponding to a set of state parameters and correlated with causing a particular physiological effect.

18. The method of claim 16, further including outputting, using the machine learning model, at least one of:
a predicted effect of the neuromodulation signal or an additional neuromodulation signal;
the event or an additional event to time the neuromodulation signal or an additional neuromodulation signal; and
a stimulus program including an additional plurality of neuromodulation signals and events to achieve an effect.

19. The method of claim 13, further including receiving data, from sensor circuitry or other communication circuitry, indicative of the occurrence of the event and, in response, determining the event has occurred and applying the neuromodulation signal within a threshold time of the event occurrence.

20. A non-transitory computer-readable storage medium comprising instructions that when executed cause processor circuitry to:
determine occurrence of an event associated with a subject; and
cause stimulation circuitry to output a neuromodulation signal to a nerve target of the subject as timed with the event, and in response, cause alteration to midbrain dopamine signals to the subject.
